SEQUENCE LISTING (14 sequences)

SEQ ID NO: 1 - DNA, 1200 bp, Campylobacter jejuni
atggctaaagaaaaattttcacgtaataagccacacgtaaatattggtactattggtcacgttgaccatggtaaaactactttaacagctgctatttctgctgttctttctagaagaggtttagcagagcttaaagattatgataatatcgataatgctccagaagaaaaagagcgtggtattactattgctacttctcatattgaatatgaaacagacaatcgtcactatgcacacgttgactgcccaggtcacgcagattatgttaaaaacatgattacaggtgctgcacaaatggatggagcgatcttggttgtttctgctgcagatggccctatgccacaaactagagagcatattcttctttctcgtcaagtaggcgttccatatattgttgtttttatgaataaagcagatatggttgatgatgctgaacttttagagttagttgaaatggaaattagagaattattaagctcttatgatttcccaggcgatgatacacctattatttctggttctgctttaaaagctcttgaagaagctaaagctggacaagatggtgaatggtcagcaaaaattatggatcttatggctgcagttgatagctatattccaactccaactcgtgatactgaaaaagacttcttgatgccaatagaagacgttttctcaatttcaggtcgtggtactgttgttacaggtagaattgaaaaaggtgttgtaaaagtaggtgatactatcgaaatcgttggtattaaagatactcaaacaacaactgtaacaggtgttgaaatgttcagaaaagaaatggatcaaggcgaagcaggagataacgtaggtgttcttcttcgtggtactaaaaaagaagaagttatccgtggtatggttcttgctaaaccaaaatcaattactccacacactgacttcgaagctgaagtttatatcttaaataaagatgaaggtggtagacatactccattctttaacaactatagaccacagttttatgtaagaacaactgatgttacaggttcgattaaattagctgatggtgttgaaatggttatgccaggtgaaaatgtgagaattactgtaagcttgatcgctccagtagcacttgaagaaggaactcgttttgctattcgtgaaggtggtaaaactgttggttcaggtgttgtttctaaaattattaaataa

SEQ ID NO: 2 - DNA, 1200 bp, Campylobacter jejuni
atggctaaagaaaaattttcacgtaataagccacacgtaaatattggtactattggtcacgttgaccatggtaaaactactttaacagctgctatttctgctgttctttctagaagaggtttagcagagcttaaagattatgataatatcgataatgctccagaagaaaaagagcgtggtattactattgctacttctcatattgaatatgaaacagacaatcgtcactatgcacacgttgactgcccaggtcacgcagattatgttaaaaacatgattacaggtgctgcacaaatggatggagcgatcttggttgtttctgctgcagatggtcctatgccacaaactagagaacacattcttctttctcgtcaagtaggcgttccatatattgttgtttttatgaataaagcagatatggttgatgatgctgaacttctagagttagttgaaatggaaattagagaattattaagctcttatgatttcccaggcgatgatacacctattatttctggttctgctttaaaagctcttgaagaagctaaagctggacaagatggtgaatggtcagcaaaaattatggatcttatggctgcagttgatagctatattccaactccaactcgtgatactgaaaaagacttcttaatgccaatagaagacgttttctcaatttcaggtcgtggtactgttgttacaggtagaattgaaaaaggtgttgtaaaagtaggtgatactatcgaaatcgttggtattaaagatactcaaacaacaactgtaacaggtgttgaaatgttcagaaaagaaatggatcaaggcgaagcaggagataacgtaggtgttcttcttcgtggtactaaaaaggaagaagttatccgtggtatggttcttgctaaaccaaaatcaattactccacacactgacttcgaagctgaagtttatatcttaaataaagatgaaggtggtagacatactccattctttaacaactatagaccacagttttatgtaagaacaactgatgttacaggttcgattaaattagctgatggtgttgaaatggttatgccaggtgaaaatgtgagaattactgtaagcttgatcgctccagtagcacttgaagaaggaactcgttttgctattcgtgaaggtggtaaaactgttggttcaggtgttgtttctaaaattattaaataa

SEQ ID NO: 3 - DNA, 1200 bp, Campylobacter jejuni
atggctaaagaaaaattttcacgtaataagccacacgtaaatattggtactattggtcacgttgaccatggtaaaactactttaacagctgctatttctgctgttctttctagaagaggtttggcagagcttaaagattatgataatatcgataatgctccagaagaaaaagagcgtggtattactattgctacttctcatattgaatatgaaacagacaatcgtcactatgcacacgttgactgcccaggtcacgcagattatgttaaaaacatgattacaggtgctgcacaaatggatggagcgatcttggttgtttctgctgcagatggccctatgccacaaactagagagcacattcttctttctcgtcaagtaggtgttccatatattgttgtttttatgaataaagcagatatggttgatgatactgaacttctagagttagttgaaatggaaattagagaattattaagctcttatgatttcccaggcgatgatacacctattatttctggttctgctttaaaagctcttgaagaagctaaagctggacaagatggtgaatggtcagcaaaaattatggatcttatggctgcagttgatagctatattccaactccaactcgtgatactgaaaaagacttcttgatgccaattgaagatgttttctcaatttcaggtcgtggtactgttgttacaggtagaattgaaaaaggtgttgtaaaagtaggtgatactatcgaaatcgttggtattaaagatactcaaacaacaactgtaacaggtgttgaaatgttcagaaaagaaatggatcaaggcgaagcaggagataacgtaggtgttcttcttcgtggtactaaaaaagaagaagttatccgtggtatggttcttgctaaaccaaaatcaattactccacacactgacttcgaagctgaagtttatatcttaaataaagatgaaggtggtagacatactccattctttaacaactatagaccacagttttatgtaagaacaactgatgttacaggttcgattaaattagctgatggtgttgaaatggttatgccaggtgaaaatgtgagaattactgtaagcttgatcgctccagtagcacttgaagaaggaactcgttttgctattcgtgaaggtggtaaaactgttggttcaggtgttgtttctaaaattattaaataa

SEQ ID NO: 4 - DNA, 1200 bp, Campylobacter jejuni; identical to SEQ ID NO: 1

SEQ ID NO: 5 - DNA, 1200 bp, Campylobacter jejuni
atggctaaagaaaaattttcacgtaataagccacacgtaaatattggtactattggtcatgttgaccatggtaaaactactttaacagctgctatttctgctgttctttctagaagaggtttagcagagcttaaagattatgataatatcgataatgctccagaagaaaaagagcgtggtattactattgctacttctcatattgaatatgaaacagacaatcgtcactatgcacacgttgactgcccaggtcacgcagattatgttaaaaacatgattacaggtgctgcacaaatggatggagcgatcttggttgtttctgctgcagatggccctatgccacaaactagagagcacattcttctttctcgtcaagtaggcgttccatatattgttgtttttatgaataaagcagatatggttgatgatgctgaacttttagagttagttgaaatggaaattagagaattattaagctcttatgatttcccaggcgatgatacacctattatttctggttctgctttaaaagctcttgaagaagctaaagctggacaagatggtgaatggtcagcaaaaattatggatcttatggctgcagttgatagctatattccaactccaactcgtgatactgaaaaagacttcttgatgccaattgaagatgttttctcaatttcaggtcgtggtactgttgttacaggtagaattgaaaaaggtgttgtaaaagtaggtgatactatcgaaatcgttggtattaaagatactcaaacaacaactgtaacaggtgttgaaatgttcagaaaagaaatggatcaaggcgaagcaggagataacgtaggtgttcttcttcgtggtactaaaaaagaagaagttatccgtggtatggttcttgctaaaccaaaatcaattactccacacactgacttcgaagctgaagtttatatcttaaataaagatgaaggtggtagacatactccattctttaacaactatagaccacagttttatgtaagaacaactgatgttacaggttcgattaaattagctgatggtgttgaaatggttatgccaggtgaaaatgtgagaattactgtaagcttgatcgctccagtagcacttgaagaaggaactcgttttgctattcgtgaaggtggtaaaactgttggttcaggtgttgtttctaaaattattaaataa

SEQ ID NO: 6 - DNA, 1200 bp, Campylobacter coli; identical to SEQ ID NO: 3

SEQ ID NO: 7 - protein, 399 aa, Artificial Sequence
  Description of Artificial Sequence C. jejuni 81116, HB-95-29, INP-59 EF-Tu, INP44 and C. coli D3088 EF-Tu peptide
MAKEKFSRNKPHVNIGTIGHVDHGKTTLTAAISAVLSRRGLAELKDYDNIDNAPEEKERGITIATSHIEYETDNRHYAHVDCPGHADYVKNMITGAAQMDGAILVVSAADGPMPQTREHILLSRQVGVPYIVVFMNKADMVDDAELLELVEMEIRELLSSYDFPGDDTPIISGSALKALEEAKAGQDGEWSAKIMDLMAAVDSYIPTPTRDTEKDFLMPIEDVFSISGRGTVVTGRIEKGVVKVGDTIEIVGIKDTQTTTVTGVEMFRKEMDQGEAGDNVGVLLRGTKKEEVIRGMVLAKPKSITPHTDFEAEVYILNKDEGGRHTPFFNNYRPQFYVRTTDVTGSIKLADGVEMVMPGENVRITVSLIAPVALEEGTRFAIREGGKTVGSGVVSKIIK

SEQ ID NO: 8 - DNA, 1032 bp, Campylobacter jejuni
atgaaaaaaaatattgtttttttcgaagttaaaggtgggagtgataaaggtgaagatggctatagaaaagataccatgcctatggtaaatgccttaaaagctaagggttggaatgctgaggtgattttttttgaagtgggtaaaaaagatgaaatttacaaatatgtgaaagaaaattttgatggttatgtttctcgcatcaatcctggcaatcttaaagaagaaaatgagtattttgatatgttaagaaaactttgtgccgataagcttgtaggcatgcctcatcctgatgctatgataggttatggcgctaaagatgcacttacaaaattagcagatactgatcttgttccaagcgatacttatgcttactatgatattaagacttttaaagaaaattttccaaaaagtttggctaaaggtgaaagggttttaaaacaaaatcgtggctctacaggagagggaatttggcgtgtgagtgtagagggtaatgttagtggagatagtttgcctttaaacacaaaaatcaaatgcacagaagctaaggataatcatgtagaacatagagagcttggggaatttatggatttttgtgagcaatatatcataggtgataatggtatgcttgtggatatgacttttttaccgcgcattaaagaaggtgaaatcagacttttaatgctttataacacccctgtaaatgtagtgcataaaaaaccagctgaagatgctgatgctttttctgctacgctttttagtggggcaaaatatcgctatgataagccagaagattggaaaactttggtggatatgtttttaggcgagcttcctaaggtaagggaaaaattaggcaattacgacttaccattgatttggacggctgattttattttagatactgatgaaaagggcaatgataaatatgttttaggcgagattaattgttcttgtgtaggctttacttctcatttagaacttgctgatgaagtagcttcaaatattattaatattgtaagtaaaactaaggcttag

SEQ ID NO: 9 - protein, 343 aa, Campylobacter jejuni
MKKNIVFFEVKGGSDKGEDGYRKDTMPMVNALKAKGWNAEVIFFEVGKKDEIYKYVKENFDGYVSRINPGNLKEENEYFDMLRKLCADKLVGMPHPDAMIGYGAKDALTKLADTDLVPSDTYAYYDIKTFKENFPKSLAKGERVLKQNRGSTGEGIWRVSVEGNVSGDSLPLNTKIKCTEAKDNHVEHRELGEFMDFCEQYIIGDNGMLVDMTFLPRIKEGEIRLLMLYNTPVNVVHKKPAEDADAFSATLFSGAKYRYDKPEDWKTLVDMFLGELPKVREKLGNYDLPLIWTADFILDTDEKGNDKYVLGEINCSCVGFTSHLELADEVASNIINIVSKTKA

SEQ ID NO: 10 - DNA, 930 bp, Campylobacter jejuni
ttgaaaaaatatttattttcctgtgttttagcctccattttaacccaatcagctacggctgtagaatttcaagaaggttttagtggaaatttaagcataggtgtgggtgcaagggatattaaaagtaatatttcaaccttggcaaacagtgattatctaagcagttacaatgctgataattcagactcctctttcattccttttatcggtgcagaactttactatggtaatcttatagataatgatagaatttttattaaaaactacaatggaagagatatcagcggtatagctttaggctacgaaagagcttatttagagcgttttagcacttctttttctgtaatttcctctttaagagaaaaagcttatgcaaatccttatgcaataggaaatagagaagaaactgatgttgatagatatggttttaaaatctctcaactttatgaaagtgattttgggaaatttaccacttcatatttatttagcaaaaacaaatatgataaagacactatcgcacaaagctctttaaaaagggagggatattatcacgaaattgaattaaactataattatagcttattaaacctagggttaaattatgattacaatgatgcagacggaaaagctcaaagctattcaagatatggttttagcataggaacaaatttggcttttgctaatgattacatcttcactccaaatttaaatcttagcaaatatgaagcagtaggaactgatcctatcttccacaaaaaacaagatggtaatatagttaagcttaatttaaaagttgttaaaaatcaatttttgggttataacggactttatggttttgcaaattatggcatagaaaaaagaaatagcgatataggattttatgatgaaacctatcaaattatcctaactggtataggatataaattctaa

SEQ ID NO: 11 - protein, 309 aa, Campylobacter jejuni
LKKYLFSCVLASILTQSATAVEFQEGFSGNLSIGVGARDIKSNISTLANSDYLSSYNADNSDSSFIPFIGAELYYGNLIDNDRIFIKNYNGRDISGIALGYERAYLERFSTSFSVISSLREKAYANPYAIGNREETDVDRYGFKISQLYESDFGKFTTSYLFSKNKYDKDTIAQSSLKREGYYHEIELNYNYSLLNLGLNYDYNDADGKAQSYSRYGFSIGTNLAFANDYIFTPNLNLSKYEAVGTDPIFHKKQDGNIVKLNLKVVKNQFLGYNGLYGFANYGIEKRNSDIGFYDETYQIILTGIGYKF

SEQ ID NO: 12 - protein, 620 aa, Campylobacter jejuni
MRLSKTLCMALLAGSTLLAPNVLMAMGGPSGAKIDWQIQGQIGAIKMNPYGLSPLTAIIMDNGYVLSDIKVTIVPKPNGQTISYNVNSKMAKTYGGIPIFGLYPSYLNTVKVSYTKTANGKSQKVIDEIYKITTPGVSIEPSGSTDQRGTPFENVKVLKMDPKFSDRLYLVNNAPGKQSGKGSQSVWNNPVGGAMEWDENSNVFIIDTKGEIRWYFDNDKLMNWDNIYNRGIMMGFHQNKDGALTWGFGQRYVKYDILGREIFNRKLPAAYIDFSHAMDNMQNGHYLLRVASANTLRPDGKHVRTVRDTIVEVDENGNVVDDWRLYEILDPYRSTIIKALDQGAVCLNIDASKAGKTLSDEELAKMDESDKFGDIAGTGIGRNWAHVNSVDYDPSDDSIIISSRHQSAVVKIGRDKKIKWILGAHKGWNKEFQKYLLQPVDKNGKKIVCDDDYSKCPGYENDNGGFDFTWTQHTGWRIDSKSNKRYIYISVFDNGDARGAEQPAFASQKYSRAVIYKIDQQNKTVEQIWEYGKNRGNEWFSPVTSLTQYEPDKDSIMVYSATAGMAFDLSKGVSLGEPKPEIDEFNWGAKEPSVQIQFSGSGTGYQAMPFSVDQAFNPKK

SEQ ID NO: 13 - protein, 281 aa, Mycobacterium tuberculosis
VAPAQALLLAAAGAAGSPIRARLPQRLRRSLRDHARDANPGRPVPGTIAATAVSMTVGTLVASVLPATVFEDLAYAELYSDPPGLTPLPEEAPLIARSVAKRRNEFITVRHCARIALDQLGVPPAPILKGDKGEPCWPDGMVGSLTHCAGYRGAVVGRRDAVRSVGIDAEPHDVLPNGVLDAISLPAERADMPRTMPAALHWDRILFCAKEATYKAWFPLTKRWLGFEDAHITFETDSTGWTGRFVSRILIDGSTLSGPPLTTLRGRWSVERGLVLTAIVL

SEQ ID NO: 14 - protein, 414 aa, Deinococcus radiodurans
MRRTVRLALFQFLVATLASFTPTLAAKPSMQWAVTRSATPAPSSVTTDSTRVYLVEGQRLQARRLSDGKLIWQAGTGISSPLVVERGIVYVTGRAREVFAFNATDGRKLWSTVLTGVPEQSHGGWADTLSVDHGMLLVASTRGIWGVNARTGQQRWFRELLDARGPLVQLGSITVWQVSTPLKSFTFGLQSETGREVWRVQTGATPLLQEDKYVFVTVPGSPSAYRMIDVPSGRSIRVDHNFRVGAPSGQQPAQGTPGELFVTGMEVCVRVTNGEVDRLNCVNRQQGRRTGGEADLLRQALGEHPVKVRRLLNASLPSGCVGTAVKTAVGVVLVDAPDMTKTRLSQLPSRAFACFFPVSNTLKVVPVGGQLIAIDEKGRQVWVVNVQGRVQQVIPVDGRLLVATSAELRILSWP

(19) United States
(12) Patent Application Publication
(10) Pub. No.: US 2005/0106159 A1
(43) Pub. Date: May 19, 2005

(54) Campylobacter jejuni outer membrane protein immunogenic composition

(76) Inventor: Stuart A. Thompson, Evans, GA (US)

Correspondence Address: SUTHERLAND ASBILL & BRENNAN LLP, 999 PEACHTREE STREET, N.E., ATLANTA, GA, 30309, US

(21) Appl. No.: 10/916,932

(22) Filed: Aug. 12, 2004

Related U.S. Application Data
(60) Provisional application No. 60/494,500, filed on Aug. 12, 2003.

(51) Int. Cl. A61K 39/00; A61K 48/00

(52) U.S. Cl. 424/185.1; 514/44

(57) ABSTRACT

The field of this invention is the development of therapeutic agents having immunogenic efficacy against Campylobacter. The present invention is directed to a method of producing monoclonal antibodies that are highly specific for epitopes of Campylobacter jejuni outer membrane proteins; to specific monoclonal antibodies made by using the epitopes; and to uses thereof. The invention is drawn further to immunogens comprising certain outer membrane proteins or portions thereof from C. jejuni.

### CROSS REFERENCE TO RELATED APPLICATIONS

The present invention claims the priority benefit of U.S. Provisional Patent Application Ser. No. 60/494,500 filed Aug. 12, 2003, the entire contents of which are hereby incorporated by reference.

### ACKNOWLEDGMENT OF FEDERAL RESEARCH SUPPORT

This invention was made, at least in part, with funding from the National Institutes of Health (AI58284 and AI55715). Accordingly, the United States Government has certain rights in this invention.

### BACKGROUND OF THE INVENTION

1. Field of the Invention

The field of this invention is the development of therapeutic agents having immunogenic efficacy against Campylobacter. The present invention relates generally to a method of preparing an immunogen comprising outer membrane proteins from Campylobacter jejuni, inoculating animals with the immunogen, and detecting the desired hybridoma-producing antibodies; and to a composition comprising the immunogen. The invention is drawn further to hybridoma cell lines developed by this method to produce monoclonal antibodies specific to Campylobacter jejuni, and uses thereof.

2. Background Art

Campylobacter jejuni is the leading cause of bacterial gastroenteritis in the U.S., and has been classified by the NIH as a Category B Bioterrorism Agent due to its ability to cause food-borne and water-borne outbreaks. There are approximately 2.4 million cases of C. jejuni disease in the U.S. annually, with an incidence exceeding that of Salmonella and Shigella combined (Labigne-Roussel, et al., 1988 J Bacteriol., 170:1704cases of C. jejuni disease in the U.S. annually, with an incidence exceeding that of Salmonella and Shigella combined (Labigne-Roussel, et al., 1988 J Bacteriol., 170:1704-1708). C. jejuni is responsible for 90-95% of Campylobacter disease, while another Campylobacter species, C. coli, is responsible for the remaining 5-10% of cases. C. jejuni infection is also the most common antecedent event to the development of Guillain-Barré Syndrome (GBS), an acute motor paralysis that apparently results from an autoimmune response directed against C. jejuni surface antigens. Reactive arthritis is also a common event following Campylobacter infection, however the pathogenesis of reactive arthritis is unknown (Skirrow & Blaser, 2000 p. 69-88, In Nachamkin & Blaser (ed.), Campylobacter, 2nd ed. ASM press, Washington, D.C.). The development of an effective immunogenic composition against C. jejuni is therefore highly desirable, in order to protect the population from both naturally occurring C. jejuni disease and disease arising from potential bioterrorist attacks.

Immunogenic compositions based on C. jejuni whole-cell preparations have been proposed, however, due to uncertainties concerning the development of GBS, alternative approaches are warranted. Killed whole-cell immunogenic compositions have shown some promise in protecting against challenge with the homologous C. jejuni strain. A mixture of formalin- and heat-killed C. jejuni 81-176, orally administered with E. coli heat labile enterotoxin (LT) as an adjuvant, elicited a vigorous mucosal and serum immune response in mice (Baqar et al., 1995 Infect Immun., 63:3731-3735). Following oral challenge with 81-176, these mice showed substantial resistance to colonization and systemic spread (Baqar et al., 1995 Infect Immun., 63:3731-3735). The efficacy of this type of immunogenic composition was further substantiated using a murine oral immunization-intranasal challenge model, which showed protection for up to 8 months post-vaccination (Baqar et al., 1996 Infect Immun., 64:4933-4939; Scott & Tribble, 2000 p. 303-319, In Nachamkin & Blaser (ed.), Campylobacter, 2nd ed. ASM press, Washington, D.C.). Experiments in non-human primates supported the immunogenicity of the killed whole-cell immunogenic composition (Baqar et al., 1995 Vaccine, 13:22-28), however, protection has not yet been reported. The protective efficacy of a killed whole-cell immunogenic composition against heterologous, antigenically distinct Campylobacter strains has not been assessed in any animal model.

The use of live attenuated immunogenic compositions has been postulated for several organisms, including C. jejuni. The advantage of such an immunogenic composition is that a person could be immunized with a wide range of native C. jejuni antigens without basis for a putative attenuated C. jejuni immunogenic composition (Scott & Tribble, 2000 p. 303-319, In Nachamkin & Blaser (ed.), Campylobacter, 2nd ed. ASM press, Washington, D.C.).

The two major issues impacting the utility of whole-cell immunogenic compositions (either killed or live attenuated) are whether these immunogenic compositions are safe and whether or not they offer broad protection against heterologous strains. First and most importantly, an immunogenic composition must not generate post-vaccination sequelae such as GBS or reactive arthritis. Second, for an immunogenic composition to be widely effective and useful, it must generate protection against the maximal number of the countless C. jejuni strains circulating in the U.S. and elsewhere in the world. For these reasons, a subunit immunogenic composition based on defined protective proteins has the highest probability of being both safe and widely protective.

An immunogenic composition consisting of highly conserved outer membrane proteins may therefore hold the most promise for safely inducing protective immunity without the potential for inducing GBS or other sequelae.

It is well recognized that C. jejuni strains are highly variable, and this will certainly impact on the development of a protein subunit immunogenic composition. For example, a recent study showed that 21% of the genes in the sequenced C. jejuni strain were either absent or highly divergent in other C. jejuni isolates (Dorrell et al., 2001 Genome Res., 11: 1706-1715); many of these dispensable loci were related to the very surface structures that are likely components of a Campylobacter immunogenic composition. Flagellin has been advanced as a Campylobacter immunogenic composition candidate (Lee et al., 1999 Infect Immun., 67:5799-5805). However, while this protein is very antigenic and has shown (limited) protection in a mouse model, it is also both antigenically variable and phase variable. Inspection of GenBank FlaA peptide entries shows that even “conserved” FlaA amino acids 5-337 exhibit as much as 10-22% amino acid sequence divergence among different strains (Lee et al., 1999 Infect Immun., 67:5799-5805). Consequently, despite its promise in protection against heterologous C. jejuni strains, a subunit immunogenic composition based solely on FlaA is not likely to be the complete answer. Therefore, it is not an ideal immunogenic composition candidate since it may not be widely protective.

Other C. jejuni proteins have been proposed as immunogenic composition candidates as well. CDT (cytolethal distending toxin) is a potential virulence factor composed of three subunits, and the genes encoding this toxin (cdtA, cdtB, and cdtC) are highly conserved among distinct C. jejuni strains (Pickett et al., 1996 Infect Immun., 64:2070-2078; Scott & Tribble, 2000 p. 303-319, In Nachamkin & Blaser (ed.), Campylobacter, 2nd ed. ASM press, Washington, D.C.). Although it has been proposed as an immunogenic composition candidate, the role of CDT in pathogenesis and its potential role as a protective antigen have yet to be demonstrated (Scott & Tribble, 2000 p. 303-319, In Nachamkin & Blaser (ed.), Campylobacter, 2nd ed. ASM press, Washington, D.C.). PEB1 is a conserved protein that is immunogenic in humans (Pei & Blaser, 1993 J Biol. Chem., 268:18717-18725), and PEB1 mutants exhibit reduced interaction with epithelial cells as well as reduced colonization of mice (Pei et al., 1998 Infect Immun., 66:938-943). Recombinant PEB1 allowed protection against colonization and disease in the mouse intranasal challenge model (Scott & Tribble, 2000 p. 303-319, In Nachamkin & Blaser (ed.), Campylobacter, 2nd ed. ASM press, Washington, D.C.).

Another factor that compounds the problems associated with the development of a Campylobacter immunogenic composition is the extreme diversity of C. jejuni strains, and the large number of phase variable proteins in C. jejuni. C. jejuni can be categorized by either of two common typing schemes. Penner serotyping distinguishes C. jejuni strains by heat stable (HS) antigens and detects >60 serotypes (Penner & Hennessy, 1980 J Clin Microbiol., 12:732-737); Lior serotyping is based on heat-labile antigens and recognizes >100 distinct serotypes (Lior et al., 1982 J Clin Microbiol., 15:761-768). There are also significant differences in disease severity, and campylobacteriosis can present as a spectrum of diseases ranging from mild, watery diarrhea to a severe dysentery-like illness with profuse diarrhea containing leukocytes and blood (Allos & Blaser, 1995 Clin Infect Dis., 20:1092-1099). Disease severity correlates in some instances with differences in the ability of a strain to adhere to and invade host cells (Everest et al., 1992 J Med Microbiol., 37:319-325; Hu & Kopecko, 1999 Infect Immun., 67:4171-4182; Hu & Kopecko, 2000, p. 191-215, In Nachamkin & Blaser (ed.), Campylobacter, 2nd ed. ASM press, Washington, D.C.; Oelschlaeger et al., 1993 Proc Natl Acad Sci USA, 90:6884-6888), although this process is poorly understood. Plasmids may also play a role in the differing virulence properties of C. jejuni, such as a putative 37 kb virulence plasmid found in approximately 10% of C. jejuni strains, including 81-176 (Bacon et al., 2000 Infect Immun., 68:4384-4390; Bacon et al., 2002 Infect Immun., 70:6242-6250). C. jejuni chromosomes also exhibit overall variability. Microarray experiments reporting substantial interstrain variability in the presence of C. jejuni chromosomal genes have been performed by two groups (Dorrell et al., 2001 Genome Res., 11: 1706-1715; Gaynor & Falkow, 2001 Int. J. Med. Microbiol., 291 Supp. 31:1-168). In particular, Dorrell et al. reported that as many as 21% of the genes present in the NCTC11168 genome were absent in other strains, and that the majority of highly variable genes encoded surface structures (Dorrell et al., 2001 Genome Res., 11:1706-1715).

Even proteins that are present in many or most strains show high antigenic variability in different strains. Examples of this include FlaA and FlaB flagellins (Caldwell et al., 1985 Infect Immun., 50:941-943; Harris et al., 1987 J Bacteriol., 169:5066-5071; Logan et al., 1989 Infect Immun., 57:2583-2585; Meinersmann & Hiett, 2000 Microbiology, 146:2283-2290), the flagellar hook protein FlgE (Lüneberg et al., 1998 J Bacteriol., 180:3711-3714), and OmpH1 (Meinersmann, 2000 p. 351-368, In Nachamkin & Blaser (ed.), Campylobacter, 2nd ed. ASM Press, Washington, D.C.; Meinersmann et al., 1997 Curr Microbiol., 34:360-366). Phase variability of proteins is extremely common in C. jejuni (Parkhill et al., 2000 Nature, 403:665-668; Wassenaar et al., 2002 FEMS Microbiol Lett., 212:77-85), so the ability of C. jejuni to switch the expression of proteins between “on” and “off” phases is an additional source of variability. Unlike the majority of bacterial proteins, some C. jejuni proteins (including flagellin) are also glycosylated (Szymanski et al, 1999 Mol Microbiol., 32:1022-1030).

The implication of the extreme diversity of C. jejuni strains is that there appear to be multiple mechanisms of pathogenesis and disease, and that these result from a greatly differing gene complement in different C. jejuni strains. The fact that each C. jejuni strain possesses a different set of proteins complicates the development of subunit immunogenic compositions, which ideally rely on proteins that are conserved among most or all strains. The understanding of these interstrain differences in protein expression is extremely lacking, and must increase significantly if the design of a rational subunit immunogenic composition is to proceed.

Most of what is known about C. jejuni outer membrane proteins is derived from the sequence of the NCTC1168 genome (Parkhill et al., 2000 Nature, 403:665-668). The genome sequence predicts <20 genes encoding outer membrane proteins. However, prediction of outer membrane protein genes by the genome sequence is only a starting point toward the understanding of outer membrane proteins that are actually present. First, the NCTC11168 genome sequence is that of only a single strain, and certainly does not represent all or even most C. jejuni strains, since C. jejuni as a species is highly variable. Consequently, NCTC11168 may have outer membrane proteins that are not found in other C. jejuni strains, and other strains may have outer membrane proteins not found in NCTC11168. A prominent example of this is strain 81-176, whose virulence plasmid (not found in NCTC11168) expresses predicted outer membrane proteins (Bacon et al., 2000 Infect Immun., 68:4384-4390; Bacon et al., 2002 Infect Immun., 70:6242-6250).

Second, only a handful of C. jejuni proteins have actually been experimentally localized to the outer membrane. MOMP is the major porin of C. jejuni (De et al., 2000 FEBS Lett., 469:93-97; Labesse et al., 2001 Biochem Biophys Res Commun., 280:380-387; Zhang et al., 2000 Infect Immun., 68:5679-5689), although an alternate porin (Omp50) has also been identified (Bolla et al., 2000 Biochem J., 352:637-643). Flagella (composed of FlaA and FlaB) are anchored to the outer membrane (Guerry, 2000 p. 405-421, In Nachamkin & Blaser (ed.), Campylobacter, 2nd ed. ASM Press, Washington, D.C.) by the OMP FlgE, the flagellar hook protein that connects the flagellar filament to the basal body (Kinsella et al., 1997 J Bacteriol., 179:4647-4653; Lüneberg et al., 1998 J Bacteriol., 180:3711-3714). The fibronectin-binding protein CadF (Konkel et al., 1997 Mol Microbiol., 24:953-963; Monteville & Konkel, 2002 Infect Immun., 70:6665-6671; Moser et al., 1997 FEMS Microbiol Lett., 157:233-238), PEB1 (Pei & Blaser, 1993 J Biol. Chem., 268:18717-18725; Pei et al., 1998 Infect Immun., 66:938-943), and JlpA (Jin et al., 2001 Mol Microbiol., 39:1225-1236) have been identified as cell surface adhesins. Other known outer membrane proteins are CmeC, the outer membrane component of the CmeABC multidrug efflux pump (Lin et al., 2002 Antimicrob Agents Chemother., 46:2124-2131), Omp18 (Burnens et al., 1995 J Clin Microbiol., 33:2826-2832; Konkel et al., 1996 Infect Immun., 64:1850-1853), and OmpH1 (Meinersmann et al., 1997 Curr Microbiol., 34:360-366), although these have not been studied in detail. Several of these outer membrane proteins are already known to be variable among different C. jejuni strains (Caldwell et al., 1985 Infect Immun., 50:941-943; Harris et al., 1987 J Bacteriol., 169:5066-5071; Logan et al., 1989 Infect Immun., 57:2583-2585; Lüneberg et al., 1998 J Bacteriol., 180:3711-3714; Pawelec et al., 2000 FEMS Microbiol Lett., 185:43-49).

There is therefore a need in the art for immunogens and immunogenic compositions that are effective against C. jejuni and optionally C. coli infection, where the immunogen is not phase variable or antigenically variable, and wherein the use of such a immunogenic composition preferably does not induce GBS or other sequelae. A protein appropriate for immunogenic composition inclusion should be conserved in the largest possible proportion of C. jejuni strains, should be immunogenic, and should induce protective immunity against a large number of diverse C. jejuni strains. While analysis of the C. jejuni genome sequence is helpful as a starting point toward understanding its complement of outer membrane proteins, only direct identification of outer membrane proteins by proteome analysis will provide detailed information about the outer membrane proteins actually expressed by C. jejuni strains.

### SUMMARY OF THE INVENTION

It is an object of the present invention to overcome, or at least alleviate, one or more of the difficulties or deficiencies associated with the prior art. In that regard, the present invention provides for an immunogen comprising one or more epitopes from an outer membrane protein that is highly conserved in multiple strains of C. jejuni and C. coli. Preferably, the immunogen induces an immune response in an animal. Preferably, the animal is a mammal, such as a mouse or a human.

The immunogen of the present invention can be used to prepare a monoclonal antibody, wherein the monoclonal antibody is specific to an epitope on C. jejuni and/or C. coli. The invention further provides for methods of treating animals comprising the administration of the immunogen or the monoclonal antibody to the animal, wherein the immunogen decreases the rate of subsequent infection by C. jejuni and/or C. coli.

The present invention encompasses an immunogenic composition comprising a polypeptide encoded by a polynucleotide sequence as defined in any one of (a) SEQ ID NO:1, SEQ ID NO:2, SEQ ID NO:3, SEQ ID NO:4, SEQ ID NO:5, SEQ ID NO:6, SEQ ID NO:8, or SEQ ID NO:10; (b) a polynucleotide sequence encoding the amino acid sequence of SEQ ID NO:7; (c) a polynucleotide sequence encoding the amino acid sequence of SEQ ID NO:9; (d) a polynucleotide sequence encoding the amino acid sequence of SEQ ID NO:11; (e) a polynucleotide sequence encoding the amino acid sequence of SEQ ID NO:12; (f) a polynucleotide sequence encoding the amino acid sequence of SEQ ID NO:13; (g) a polynucleotide sequence encoding the amino acid sequence of SEQ ID NO:14; (h) a polynucleotide of at least 50 consecutive nucleotides of any of (a)-(g); and (i) an ortholog or homolog of any of (a)-(g). The invention further encompasses an immunogenic composition comprising a purified polypeptide as defined in any one of (a) SEQ ID NO:7, SEQ ID NO:9, SEQ ID NO:11, SEQ ID NO:12, SEQ ID NO:13, or SEQ ID NO:14; (b) a 50 amino acid fragment of any of (a); and (c) an ortholog or homolog of any of a).

The invention is further directed towards a live bacterial cell vector that (a) infects a human, and (b) is stably transformed with, and expresses, a heterologous DNA encoding a Campylobacter outer membrane protein antigen, operatively associated with a regulatory sequence that controls gene expression. In certain embodiments, the heterologous DNA encodes antigen EF-Tu. Preferably, the heterologous DNA encoding the EF-Tu antigen encodes an amino acid as defined in SEQ ID NO:7, a consecutive 50 amino acid fragment thereof; or an ortholog or homolog thereof. In other embodiments, the heterologous DNA encodes antigen Cj0069. Preferably, the heterologous DNA encoding the Cj0069 antigen encodes an amino acid as defined in SEQ ID NO:9, a consecutive 50 amino acid fragment thereof; or an ortholog or homolog thereof. In yet another embodiment, the heterologous DNA encodes antigen Cj0561c. Preferably, the heterologous DNA encoding the Cj0561c antigen encodes an amino acid as defined in SEQ ID NO:11, a consecutive 50 amino acid fragment thereof; or an ortholog or homolog thereof. In another embodiment, the heterologous DNA encodes antigen AstA. Preferably, the heterologous DNA encoding the AstA antigen encodes an amino acid as defined in SEQ ID NO:12, a consecutive 50 amino acid fragment thereof; or an ortholog or homolog thereof. In a further embodiment, the heterologous DNA encodes antigen Rv2794c. Preferably, the heterologous DNA encoding the Rv2794c antigen encodes an amino acid as defined in SEQ ID NO:13, a consecutive 50 amino acid fragment thereof; or an ortholog or homolog thereof. In another embodiment, the heterologous DNA encodes antigen DRC0015. Preferably, the heterologous DNA encoding the DRC0015 antigen encodes an amino acid as defined in SEQ ID NO:14, a consecutive 50 amino acid fragment thereof; or an ortholog or homolog thereof.

The invention further encompasses a method of eliciting an immune response in an animal, comprising introducing into the animal a composition comprising a purified polypeptide encoded by a polynucleotide sequence as defined in any one of (a) SEQ ID NO:1, SEQ ID NO:2, SEQ ID NO:3, SEQ ID NO:4, SEQ ID NO:5, SEQ ID NO:6, SEQ ID NO:8, or SEQ ID NO:10; (b) a polynucleotide sequence encoding the amino acid sequence of SEQ ID NO:7; (c) a polynucleotide sequence encoding the amino acid sequence of SEQ ID NO:9; (d) a polynucleotide sequence encoding the amino acid sequence of SEQ ID NO:11; (e) a polynucleotide sequence encoding the amino acid sequence of SEQ ID NO:12; (f) a polynucleotide sequence encoding the amino acid sequence of SEQ ID NO:13; (g) a polynucleotide sequence encoding the amino acid sequence of SEQ ID NO:14; (h) a polynucleotide of at least 50 consecutive nucleotides of any of (a)-(g); and (i) an ortholog or homolog of any of (a)-(g).

The invention is further directed to a method of generating antibodies specific for antigen EF-Tu, comprising introducing into an animal a composition comprising a purified polypeptide encoded by a polynucleotide sequence as defined in any one of (a) SEQ ID NO:1, SEQ ID NO:2, SEQ ID NO:3, SEQ ID NO:4, SEQ ID NO:5, or SEQ ID NO:6; (b) a polynucleotide sequence encoding the amino acid sequence of SEQ ID NO:7; (c) a polynucleotide of at least 50 consecutive nucleotides of any of (a) or (b); and (d) an ortholog or homolog of any of (a) or (b). The invention is also directed to a method of generating antibodies specific for antigen Cj0069, comprising introducing into an animal a composition comprising a purified polypeptide encoded by a polynucleotide sequence as defined in any one of (a) SEQ ID NO:8; (b) a polynucleotide sequence encoding the amino acid sequence of SEQ ID NO:9; (c) a polynucleotide of at least 50 consecutive nucleotides of any of (a) or (b); and (d) an ortholog or homolog of any of (a) or (b). The invention also encompasses a method of generating antibodies specific for antigen Cj0561c, comprising introducing into an animal a composition comprising a purified polypeptide encoded by a polynucleotide sequence as defined in any one of (a) SEQ ID NO:10; (b) a polynucleotide sequence encoding the amino acid sequence of SEQ ID NO:11; (c) a polynucleotide of at least 50 consecutive nucleotides of any of (a) or (b); and (d) an ortholog or homolog of any of (a) or (b). In yet another embodiment, the invention encompasses a method of generating antibodies specific for antigen AstA, comprising introducing into an animal a composition comprising a purified polypeptide as defined in the amino acid sequence of SEQ ID NO:12, a consecutive 50 amino acid fragment thereof, or an ortholog or homolog thereof. It also encompasses a method of generating antibodies specific for antigen Rv2794c, comprising introducing into an animal a composition comprising a purified polypeptide as defined in the amino acid sequence of SEQ ID NO:13, a consecutive 50 amino acid fragment thereof, or an ortholog or homolog thereof. In a further embodiment, the invention encompasses a method of generating antibodies specific for antigen DRC0015, comprising introducing into an animal a composition comprising a purified polypeptide as defined in the amino acid sequence of SEQ ID NO:14, a consecutive 50 amino acid fragment thereof, or an ortholog or homolog thereof.

In certain embodiments, the methods further comprise detecting the presence of antibodies specific for the antigen in the animal. It is preferred that the animal used for generating the antibodies is susceptible to infection with Campylobacter. In one embodiment, the amount of the purified polypeptide introduced into the animal is sufficient to induce an immune response protective against Campylobacter infection.

The invention further encompasses a purified antibody that binds specifically to a protein selected from the group consisting of EF-Tu, Cj0069, Cj0561c, AstA, Rv2794c, and DRC0015. Preferably, the antibody is selected from the group consisting of recombinant antibodies, humanized chimeric antibodies and immunologically active fragments of antibodies.

It is also contemplated that the invention is directed to a method of making an antibody, comprising immunizing a non-human animal with an immunogenic fragment of a protein selected from a group consisting of EF-Tu, Cj0069, Cj0561c, AstA, Rv2794c, and DRC0015. In other embodiments, the method of making an antibody comprises providing a hybridoma cell that produces a monoclonal antibody specific for a protein selected from a group consisting of EF-Tu, Cj0069, Cj0561c, AstA, Rv2794c, and DRC0015, and culturing the cell under conditions that permit production of the monoclonal antibody.

The invention is also directed to a method of inhibiting Campylobacter infection in a patient, comprising administering to the patient a composition a purified antibody that binds specifically to a protein selected from a group consisting of EF-Tu, Cj0069, Cj0561c, AstA, Rv2794c, and DRC0015, wherein the antibody is selected from the group consisting of recombinant antibodies, humanized chimeric antibodies and immunologically active fragments of antibodies. Preferably, the administration of the purified antibody inhibits Campylobacter infection by decreasing the rate of subsequent infection by C. jejuni and/or C. coli.

In various embodiments, the invention encompasses a method of determining whether a biological sample contains C. jejuni, comprising contacting the sample an antibody specific for a protein selected from a group consisting of EF-Tu, Cj0069, Cj0561c, AstA, Rv2794c, and DRC0015 and determining whether the antibody specifically binds to the sample, said binding being an indication that the sample contains C. jejuni.

The invention is also directed to a method of purifying a protein from a biological sample containing a protein selected from a group consisting of EF-Tu, Cj006, Cj0561c, AstA, Rv2794c, and DRC0015, comprising (a) providing an affinity matrix comprising an antibody specific for a protein selected from a group consisting of EF-Tu, Cj0069, Cj0561c, AstA, Rv2794c bound to a solid support; (b) contacting the biological sample with the affinity matrix, to produce an affinity matrix-protein complex; (c) separating the affinity matrix-protein complex from the remainder of the biological sample; and (d) releasing the protein from the affinity matrix. In one embodiment, the protein purified from the affinity matrix comprises the amino acid sequence as defined in SEQ ID NO:7, or an ortholog or homolog thereof. In another embodiment, the protein purified from the affinity matrix comprises the amino acid sequence as defined in SEQ ID NO:9, or an ortholog or homolog thereof. In a further embodiment, the protein purified from the affinity matrix comprises the amino acid sequence as defined in SEQ ID NO:11, or an ortholog or homolog thereof. In yet another embodiment, the protein purified from the affinity matrix comprises the amino acid sequence as defined in SEQ ID NO:12, or an ortholog or homolog thereof. In another embodiment, the protein purified from the affinity matrix comprises the amino acid sequence as defined in SEQ ID NO:13, or an ortholog or homolog thereof. In a further embodiment, the protein purified from the affinity matrix comprises the amino acid sequence as defined in SEQ ID NO:14, or an ortholog or homolog thereof.

### BRIEF DESCRIPTION OF THE DRAWINGS

FIG. 1 depicts a 2-dimensional SDS-PAGE gel of C. jejuni 81-176 outer membrane proteins. The arrows and numbers indicate proteins spots that were picked and analyzed by mass spectrometry.

FIG. 2 provides a list of proteins from a C. jejuni outer membrane fraction as identified by mass spectrometry. The numbers of the proteins correspond to the numbers of the protein spots in FIG. 1.

FIGS. 3A-F show the nucleotide sequence of C. jejuni 81-176 EF-Tu (tufB gene; SEQ ID NO:1); the nucleotide sequence of C. jejuni 81116 EF-Tu (tufB gene; SEQ ID NO:2); the nucleotide sequence of C. jejuni HB-95-29 EF-Tu (tufB gene; SEQ ID NO:3); the nucleotide sequence of C. jejuni INP-59 EF-Tu (tufB gene; SEQ ID NO:4); the nucleotide sequence of C. jejuni INP44 EF-Tu (tufB gene; SEQ ID NO:5); and the nucleotide sequence of C. coli D3088 EF-Tu (tufB gene; SEQ ID NO:6). FIG. 3G shows the amino acid sequence of C. jejuni 81116, HB-95-29, INP-59 EF-Tu, INP44 and C. coli D3088 EF-Tu (SEQ ID NO:7).

FIGS. 4A-B show the nucleotide sequence of the C. jejuni 81-176 ortholog of Cj0069 (SEQ ID NO:8); and the amino acid sequence of the C. jejuni 81-176 ortholog of Cj0069 (SEQ ID NO:9).

FIGS. 5A-B show the nucleotide sequence of the C. jejuni 81-176 ortholog of Cj0561 (SEQ ID NO:10); and the amino acid sequence of the C. jejuni 81-176 ortholog of Cj0561c (SEQ ID NO:11).

FIG. 6 shows the amino acid sequence of C. jejuni Arylsulfatase (SEQ ID NO:12).

FIG. 7 shows the amino acid sequence of M. tuberculosis Rv2794c (SEQ ID NO:13).

FIG. 8 shows the amino acid sequence of D. radiodurans DRC0015 (SEQ ID NO:14).

### DETAILED DESCRIPTION OF THE INVENTION

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

This invention relates to the generation of biologically active Campylobacter proteins or protein fragments for use in immunogenic compositions and methods of providing protective immunity to animals, including humans, against Campylobacter infection or disease. As used herein, an “immunogenic composition” is capable of generating an immune response in the animal to which it is administered. An immune response includes either or both of a cellular immune response or production of antibodies, and can include activation of the subject's B cells, T cells, helper T cells or other cells of the subject's immune system. Immunogenicity of C. jejuni outer membrane proteins or protein fragments can be determined, for example, by administering the adjuvanted protein or protein fragment to the subject, then observing the associated immune response by analyzing antibody titers in the subject's serum. This immune response may interfere with the infectivity or activity of the Campylobacter species, or it may limit the spread or reproduction of the bacteria. The immune response resulting from treatment with an immunogenic composition containing the proteins or protein fragments of the present invention provides protection against subsequent challenge by a homologous or heterologous Campylobacter species.

The present invention encompasses an immunogenic composition comprising a polypeptide encoded by a polynucleotide sequence as defined in any one of (a) SEQ ID NO:1, SEQ ID NO:2, SEQ ID NO:3, SEQ ID NO:4, SEQ ID NO:5, SEQ ID NO:6, SEQ ID NO:8, or SEQ ID NO:10; (b) a polynucleotide sequence encoding the amino acid sequence of SEQ ID NO:7; (c) a polynucleotide sequence encoding the amino acid sequence of SEQ ID NO:9; (d) a polynucleotide sequence encoding the amino acid sequence of SEQ ID NO:11; (e) a polynucleotide sequence encoding the amino acid sequence of SEQ ID NO:12; (f) a polynucleotide sequence encoding the amino acid sequence of SEQ ID NO:13; (g) a polynucleotide sequence encoding the amino acid sequence of SEQ ID NO:14; (h) a polynucleotide of at least 50 consecutive nucleotides of any of (a)-(g); and (i) an ortholog or homolog of any of (a)-(g). The invention further encompasses an immunogenic composition comprising a purified polypeptide as defined in any one of (a) SEQ ID NO:7, SEQ ID NO:9, SEQ ID NO:11, SEQ ID NO:12, SEQ ID NO:13, or SEQ ID NO:14; (b) a 50 amino acid fragment of any of (a); and (c) an ortholog or homolog of any of a).

The invention is further directed towards a live bacterial cell vector that (a) infects a human, and (b) is stably transformed with, and expresses, a heterologous DNA encoding a Campylobacter outer membrane protein antigen, operatively associated with a regulatory sequence that controls gene expression. In certain embodiments, the heterologous DNA encodes antigen EF-Tu. Preferably, the heterologous DNA encoding the EF-Tu antigen encodes an amino acid as defined in SEQ ID NO:7, a consecutive 50 amino acid fragment thereof; or an ortholog or homolog thereof. In other embodiments, the heterologous DNA encodes antigen Cj0069. Preferably, the heterologous DNA encoding the Cj0069 antigen encodes an amino acid as defined in SEQ ID NO:9, a consecutive 50 amino acid fragment thereof; or an ortholog or homolog thereof. In yet another embodiment, the heterologous DNA encodes antigen Cj0561c. Preferably, the heterologous DNA encoding the Cj0561c antigen encodes an amino acid as defined in SEQ ID NO:11, a consecutive 50 amino acid fragment thereof; or an ortholog or homolog thereof. In another embodiment, the heterologous DNA encodes antigen AstA. Preferably, the heterologous DNA encoding the AstA antigen encodes an amino acid as defined in SEQ ID NO:12, a consecutive 50 amino acid fragment thereof; or an ortholog or homolog thereof. In a further embodiment, the heterologous DNA encodes antigen Rv2794c. Preferably, the heterologous DNA encoding the Rv2794c antigen encodes an amino acid as defined in SEQ ID NO:13, a consecutive 50 amino acid fragment thereof; or an ortholog or homolog thereof. In another embodiment, the heterologous DNA encodes antigen DRC0015. Preferably, the heterologous DNA encoding the DRC0015 antigen encodes an amino acid as defined in SEQ ID NO:14, a consecutive 50 amino acid fragment thereof; or an ortholog or homolog thereof.

The invention further encompasses a method of eliciting an immune response in an animal, comprising introducing into the animal a composition comprising a purified polypeptide encoded by a polynucleotide sequence as defined in any one of (a) SEQ ID NO:1, SEQ ID NO:2, SEQ ID NO:3, SEQ ID NO:4, SEQ ID NO:5, SEQ ID NO:6, SEQ ID NO:8, or SEQ ID NO:10; (b) a polynucleotide sequence encoding the amino acid sequence of SEQ ID NO:7; (c) a polynucleotide sequence encoding the amino acid sequence of SEQ ID NO:9; (d) a polynucleotide sequence encoding the amino acid sequence of SEQ ID NO:11; (e) a polynucleotide sequence encoding the amino acid sequence of SEQ ID NO:12; (f) a polynucleotide sequence encoding the amino acid sequence of SEQ ID NO:13; (g) a polynucleotide sequence encoding the amino acid sequence of SEQ ID NO:14; (h) a polynucleotide of at least 50 consecutive nucleotides of any of (a)-(g); and (i) an ortholog or homolog of any of (a)-(g).

The invention is further directed to a method of generating antibodies specific for antigen EF-Tu, comprising introducing into an animal a composition comprising a purified polypeptide encoded by a polynucleotide sequence as defined in any one of (a) SEQ ID NO:1, SEQ ID NO:2, SEQ ID NO:3, SEQ ID NO:4, SEQ ID NO:5, or SEQ ID NO:6; (b) a polynucleotide sequence encoding the amino acid sequence of SEQ ID NO:7; (c) a polynucleotide of at least 50 consecutive nucleotides of any of (a) or (b); and (d) an ortholog or homolog of any of (a) or (b). The invention is also directed to a method of generating antibodies specific for antigen Cj0069, comprising introducing into an animal a composition comprising a purified polypeptide encoded by a polynucleotide sequence as defined in any one of (a) SEQ ID NO:8; (b) a polynucleotide sequence encoding the amino acid sequence of SEQ ID NO:9; (c) a polynucleotide of at least 50 consecutive nucleotides of any of (a) or (b); and (d) an ortholog or homolog of any of (a) or (b). The invention also encompasses a method of generating antibodies specific for antigen Cj0561c, comprising introducing into an animal a composition comprising a purified polypeptide encoded by a polynucleotide sequence as defined in any one of (a) SEQ ID NO:10; (b) a polynucleotide sequence encoding the amino acid sequence of SEQ ID NO:11; (c) a polynucleotide of at least 50 consecutive nucleotides of any of (a) or (b); and (d) an ortholog or homolog of any of (a) or (b). In yet another embodiment, the invention encompasses a method of generating antibodies specific for antigen AstA, comprising introducing into an animal a composition comprising a purified polypeptide as defined in the amino acid sequence of SEQ ID NO:12, a consecutive 50 amino acid fragment thereof, or an ortholog or homolog thereof. It also encompasses a method of generating antibodies specific for antigen Rv2794c, comprising introducing into an animal a composition comprising a purified polypeptide as defined in the amino acid sequence of SEQ ID NO:13, a consecutive 50 amino acid fragment thereof, or an ortholog or homolog thereof. In a further embodiment, the invention encompasses a method of generating antibodies specific for antigen DRC0015, comprising introducing into an animal a composition comprising a purified polypeptide as defined in the amino acid sequence of SEQ ID NO:14, a consecutive 50 amino acid fragment thereof, or an ortholog or homolog thereof.

In certain embodiments, the methods further comprise detecting the presence of antibodies specific for the antigen in the animal. It is preferred that the animal used for generating the antibodies is susceptible to infection with Campylobacter. In one embodiment, the amount of the purified polypeptide introduced into the animal is sufficient to induce an immune response protective against Campylobacter infection.

The invention further encompasses a purified antibody that binds specifically to a protein selected from the group consisting of EF-Tu, Cj0069, Cj0561c, AstA, Rv2794c, and DRC0015. Preferably, the antibody is selected from the group consisting of recombinant antibodies, humanized chimeric antibodies and immunologically active fragments of antibodies.

It is also contemplated that the invention is directed to a method of making an antibody, comprising immunizing a non-human animal with an immunogenic fragment of a protein selected from a group consisting of EF-Tu, Cj0069, Cj0561c, AstA, Rv2794c, and DRC0015. In other embodiments, the method of making an antibody comprises providing a hybridoma cell that produces a monoclonal antibody specific for a protein selected from a group consisting of EF-Tu, Cj0069, Cj0561c, AstA, Rv2794c, and DRC0015, and culturing the cell under conditions that permit production of the monoclonal antibody.

The invention is also directed to a method of inhibiting Campylobacter infection in a patient, comprising administering to the patient a composition a purified antibody that binds specifically to a protein selected from a group consisting of EF-Tu, Cj0069, Cj0561c, AstA, Rv2794c, and DRC0015, wherein the antibody is selected from the group consisting of recombinant antibodies, humanized chimeric antibodies and immunologically active fragments of antibodies. Preferably, the administration of the purified antibody inhibits Campylobacter infection by decreasing the rate of subsequent infection by C. jejuni and/or C. coli.

In various embodiments, the invention encompasses a method of determining whether a biological sample contains C. jejuni, comprising contacting the sample an antibody specific for a protein selected from a group consisting of EF-Tu, Cj0069, Cj0561c, AstA, Rv2794c, and DRC0015 and determining whether the antibody specifically binds to the sample, said binding being an indication that the sample contains C. jejuni.

The invention is also directed to a method of purifying a protein from a biological sample containing a protein selected from a group consisting of EF-Tu, Cj006, Cj0561c, AstA, Rv2794c, and DRC0015, comprising (a) providing an affinity matrix comprising an antibody specific for a protein selected from a group consisting of EF-Tu, Cj0069, Cj0561c, AstA, Rv2794c bound to a solid support; (b) contacting the biological sample with the affinity matrix, to produce an affinity matrix-protein complex; (c) separating the affinity matrix-protein complex from the remainder of the biological sample; and (d) releasing the protein from the affinity matrix. In one embodiment, the protein purified from the affinity matrix comprises the amino acid sequence as defined in SEQ ID NO:7, or an ortholog or homolog thereof. In another embodiment, the protein purified from the affinity matrix comprises the amino acid sequence as defined in SEQ ID NO:9, or an ortholog or homolog thereof. In a further embodiment, the protein purified from the affinity matrix comprises the amino acid sequence as defined in SEQ ID NO:11, or an ortholog or homolog thereof. In yet another embodiment, the protein purified from the affinity matrix comprises the amino acid sequence as defined in SEQ ID NO:12, or an ortholog or homolog thereof. In another embodiment, the protein purified from the affinity matrix comprises the amino acid sequence as defined in SEQ ID NO:13, or an ortholog or homolog thereof. In a further embodiment, the protein purified from the affinity matrix comprises the amino acid sequence as defined in SEQ ID NO:14, or an ortholog or homolog thereof.

In a preferred embodiment of the immunogenic composition, the outer membrane protein or protein fragment used in the immunogenic composition or encoded by the polynucleotide used in the immunogenic composition further includes at least one epitope or epitope mimic, such as a T cell, helper T cell or B cell epitope or epitope mimic. Epitopes or epitope mimics can be derived from the species to which the immunogenic composition is to be administered, from the species that was the source of the polypeptide antigen or hapten, or from any other species, including a virus, bacterium, or parasite. The use of immune cell epitopes derived from an immunogenic organism, such as a pathogenic parasite, is preferred.

As used herein, a “biologically active fragment of a polypeptide” refers to a polypeptide fragment, as defined below, exhibiting at least one of the characteristics of the polypeptides according to the invention, in particular in that it is capable of eliciting an immune response directed against C. jejuni or against both C. jejuni and C. coli; and/or capable of being recognized by an antibody specific for a polypeptide according to the invention; and/or capable of binding to a polypeptide or to a nucleotide sequence of C. jejuni or of both C. jejuni and C. coli; and/or capable of modulating, regulating, inducing or inhibiting the expression of a gene of C. jejuni or of both C. jejuni and C. coli; and/or capable of modulating the replication cycle of C. jejuni or both C. jejuni and C. coli; or one of its associated microorganisms in the host cell and/or organism; and/or capable of generally exerting an even partial physiological activity, such as for example a structural activity (cellular envelope, ribosome), an enzymatic (metabolic) activity, a transport activity, an activity in the secretion or in the virulence.

The immunogenic compositions of the present invention are preferably composed of a Campylobacter outer membrane protein or immunogenic fragment(s) thereof, an adjuvant, and a pharmaceutically acceptable carrier.

The Campylobacter outer membrane protein or immunogenic fragment(s) thereof of the present invention may comprise any Campylobacter outer membrane protein or immunogenic fragment(s) thereof. The Campylobacter outer membrane protein may be chosen from any of the outer membrane proteins encoded by the Campylobacter genome.

The Campylobacter outer membrane protein or immunogenic fragment(s) of the present invention may be used in an immunogenic composition at a concentration effective to elicit an immune response from an immunized subject. The effective concentrations of Campylobacter outer membrane protein or immunogenic fragment(s) are readily determined by one of ordinary skill in the art using experimental techniques well known in that art.

The invention is also directed toward producing Campylobacter proteins for use in immunogenic compositions directed to protect immunized individuals from Campylobacter infection and/or disease. Accordingly, the invention contemplates the use of an adjuvant, such as an immunogenic protein, effective to induce desirable immune responses from an immunized animal. Such a protein should possess those biochemical characteristics required to facilitate the induction of a protective immune response from immunized vertebrates while simultaneously avoiding toxic effects to the immunized animal.

In one embodiment of the present invention, Campylobacter outer membrane proteins or fragments thereof are mixed with an adjuvant such as a bacterial toxin. The bacterial toxin may be a cholera toxin. Alternatively, the Campylobacter outer membrane proteins or fragments thereof may be mixed with the B subunit of cholera toxin (CTB). In another embodiment, an E. coli toxin may be mixed with the fusion protein. For example, the fusion protein may be mixed with E. coli heat-labile toxin (LT). The fusion proteins of the present invention may be mixed with the B subunit of E. coli heat-labile toxin (LTB) to form an immunogenic composition. Other adjuvants such as cholera toxin, labile toxin, tetanus toxin or toxoid, poly[di(carboxylatophenoxy)phosphazene] (PCPP), saponins Quil A, QS-7, and QS-21, RIBI (HAMILTON, Mont.), monophosphoryl lipid A, immunostimulating complexes (ISCOM), Syntax, Titer Max, M59, CpG, dsRNA, and CTA1-DD (the cholera toxin A1 subunit (CTA1) fused to a dimer of the Ig-binding D-region of Staphylococcus aureus protein A (DD)), are also contemplated.

The adjuvants discussed above may be used in an immunogenic composition at a concentration effective to assist in the eliciting of an immune response against the Campylobacter outer membrane proteins or fragments thereof of the present invention from an immunized subject. The effective concentrations of adjuvants may be determined by one of ordinary skill in the art using experimental techniques well known in that art.

The invention also contemplates immunization with Campylobacter outer membrane proteins or fragments thereof, and a suitable adjuvant contained in a pharmaceutically acceptable composition. Such a composition should be sterile, isotonic, and provide a non-destabilizing environment for the fusion protein and the adjuvant. Examples of this are buffers, tissue culture media, various transport media and solutions containing proteins, such as BSA, sugars, and/or polysaccharides.

The immunogenic compositions of the invention contain conventional pharmaceutical carriers. Suitable carriers are well known to those of skill in the art. These immunogenic compositions may be prepared in liquid unit dose forms. Other optional components, e.g., stabilizers, buffers, preservatives, excipients and the like may be readily selected by one of skill in the art. However, the compositions may be lyophilized and reconstituted by the individual administering the immunogenic composition prior to administration of the dose. Alternatively, the immunogenic compositions may be prepared in any manner appropriate for the chosen mode of administration, e.g., intranasal administration, oral administration, etc. The preparation of a pharmaceutically acceptable immunogenic composition, having due regard to pH, isotonicity, stability and the like, is within the skill of the art.

The dosage regimen involved in a method for eliciting an immunogenic response, including the timing, number and amounts of booster immunogenic compositions, will be determined considering various hosts and environmental factors, e.g., the age of the patient, time of administration and the geographical location and environment. The period of time for the dosing schedule is readily determined by one of skill in the art. In certain embodiments, the purified polypeptide is re-introduced after approximately 1 week, 2 weeks, 3 weeks, 4 weeks, 5 weeks, 6 weeks, 8 weeks, or 12 or more weeks.

Also included in the present invention are methods of eliciting immunogenic responses in humans to protect them against Campylobacter infection and disease with the novel Campylobacter outer membrane proteins or fragments thereof and immunogenic compositions described above. The immunogenic compositions, comprising a full-length Campylobacter outer membrane protein, a Campylobacter outer membrane fusion protein, fragments thereof, or mixtures of the above, and an adjuvant described herein may be administered by a variety of routes contemplated by the present invention. Such routes include intranasal, oral, rectal, vaginal, intramuscular, intradermal and subcutaneous administration.

Immunogenic compositions for parenteral administration include sterile aqueous or non-aqueous solutions, suspensions or emulsions, the protein immunogenic composition, and an adjuvant as described herein. The composition may be in the form of a liquid, a slurry, or a sterile solid that can be dissolved in a sterile injectable medium before use. The parenteral administration is preferably intramuscular. Intramuscular inoculation involves injection via a syringe into the muscle. This injection can be via a syringe or comparable means. The immunogenic composition may contain a pharmaceutically acceptable carrier. Alternatively, the present immunogenic composition may be administered via a mucosal route, in a suitable dose, and in a liquid form. For oral administration, the immunogenic composition can be administered in liquid, or solid form with a suitable carrier.

The calculation of appropriate doses to elicit a protective immune response using the immunogenic compositions of the present invention is well known to those of skill in the art.

A variety of immunization methods are contemplated by the invention to maximize the efficacy of the immunogenic compositions described herein. In one embodiment, females of offspring-bearing age are immunized with the immunogenic compositions of the invention. In this embodiment, immunized females develop a protective immune response directed against Campylobacter infection or disease and then passively communicate this protection to an offspring by nursing. In another embodiment, newborns are immunized with the immunogenic compositions of the invention and shortly thereafter the nursing mother is immunized with the same immunogenic composition. This two-tiered approach to vaccination provides the newborn with immediate exposure to bacterial epitopes that may themselves be protecting. Nevertheless, the passive immunity supplied by the mother would augment the protection enjoyed by the offspring. This method would therefore provide the offspring with both active and passive protection against Campylobacter infection of disease.

In still another embodiment, an individual is immunized with the immunogenic composition of the invention subsequent to immunization with a multivalent immunogenic composition. The immunization of a subject with two different immunogenic compositions may synergistically act to increase the protection an immunized individual would enjoy over that obtained with only one immunogenic composition formulation.

In other formulations of the present invention, vaccines or immunogenic compositions can also comprise DNA immunogenic or vaccine compositions comprising polynucleotide sequences of the invention operatively associated with a regulatory sequence that controls gene expression. Such compositions can include compositions that direct expression of a neutralizing epitope of Campylobacter.

The invention also comprises the use of a transformed cell according to the invention, for the preparation of an immunogenic composition.

The invention also relates to the use of DNA encoding polypeptides of Campylobacter jejuni, in particular antigenic determinants, to be formulated as immunogenic compositions. In particular, the polynucleotide sequence is as defined in any one of (a) SEQ ID NO:1, SEQ ID NO:2, SEQ ID NO:3, SEQ ID NO:4, SEQ ID NO:5, SEQ ID NO:6, SEQ ID NO:8, or SEQ ID NO:10; (b) a polynucleotide sequence encoding the amino acid sequence of SEQ ID NO:7; (c) a polynucleotide sequence encoding the amino acid sequence of SEQ ID NO:9; (d) a polynucleotide sequence encoding the amino acid sequence of SEQ ID NO:11; (e) a polynucleotide sequence encoding the amino acid sequence of SEQ ID NO:12; (f) a polynucleotide sequence encoding the amino acid sequence of SEQ ID NO:13; (g) a polynucleotide sequence encoding the amino acid sequence of SEQ ID NO:14; (h) a polynucleotide of at least 50 consecutive nucleotides of any of (a)-(g); and (i) an ortholog or homolog of any of (a)-(g). In accordance with this aspect of the invention, the DNA of interest is engineered into an expression vector under the control of regulatory elements, which will promote expression of the DNA, i.e., promoter or enhancer elements. In one preferred embodiment, the promoter element may be cell-specific and permit substantial transcription of the DNA only in predetermined cells. The DNA may be introduced directly into the host either as naked DNA (U.S. Pat. No. 5,679,647 incorporated herein by reference in its entirety) or formulated in compositions with other agents which may facilitate uptake of the DNA including viral vectors, i.e., adenovirus vectors, or agents which facilitate immunization, such as bupivicaine and other local anesthetics (U.S. Pat. No. 5,593,972 incorporated herein by reference in its entirety), saponins (U.S. Pat. No. 5,739,118 incorporated herein by reference in its entirety) and cationic polyamines (published international application WO 96/10038 incorporated herein by reference in its entirety).

The DNA sequence encoding the antigenic polypeptide and regulatory element may be inserted into a stable cell line or cloned microorganism, using techniques, such as targeted homologous recombination, which are well known to those of skill in the art, and described e.g., in Chappel, U.S. Pat. No. 4,215,051; Skoultchi, WO 91/06667 each of which is incorporated herein by reference in its entirety.

Such cell lines and microorganisms may be formulated for immunogenic composition purposes. In yet another embodiment, the DNA sequence encoding the antigenic polypeptide and regulatory element may be delivered to a mammalian host and introduced into the host genome via homologous recombination (See, Chappel, U.S. Pat. No. 4,215,051; Skoultchi, WO 91/06667 each of which is incorporated herein by reference in its entirety.

Preferably, the immunogenic compositions according to the invention intended for the prevention and/or the treatment of an infection by Campylobacter jejuni or by an associated microorganism will be chosen from the immunogenic compositions comprising a polypeptide or one of its representative fragments corresponding to a protein, or one of its representative fragments, of the outer membrane of Campylobacter jejuni. The immunogenic compositions comprising nucleotide sequences will also preferably comprise nucleotide sequences encoding a polypeptide or one of its representative fragments corresponding to a protein, or one of its representative fragments, of the outer membrane of Campylobacter jejuni.

Among these preferred immunogenic compositions, the most preferred are those comprising a polypeptide or one of its representative fragments, or a nucleotide sequence or one of its representative fragments whose sequences are chosen from the nucleotide or amino acid sequences identified and described herein. In certain embodiments, the nucleotide sequence is selected from the group consisting of the nucleotide sequence of C. jejuni 81-176 EF-Tu (tufB gene; SEQ ID NO:1); the nucleotide sequence of C. jejuni 81116 EF-Tu (tufB gene; SEQ ID NO:2); the nucleotide sequence of C. jejuni HB-95-29 EF-Tu (tufB gene; SEQ ID NO:3); the nucleotide sequence of C. jejuni INP-59 EF-Tu (tufB gene; SEQ ID NO:4); the nucleotide sequence of C. jejuni INP44 EF-Tu (tufB gene; SEQ ID NO:5); the nucleotide sequence of C. coli D3088 EF-Tu (tufB gene; SEQ ID NO:6); the nucleotide sequence of C. jejuni 81-176 ortholog of Cj0069 (SEQ ID NO:8); the nucleotide sequence of C. jejuni 81-176 ortholog of Cj0561 (SEQ ID NO:10); the nucleotide sequence encoding the amino acid sequence of C. jejuni Arylsulfatase (SEQ ID NO:12); the nucleotide sequence encoding the amino acid sequence of Mycobacterium tuberculosis Rv2794c (SEQ ID NO:13); the nucleotide sequence encoding the amino acid sequence of Deinococcus radiodurans DRC0015 (SEQ ID NO:14); and orthologs and homologs thereof. In other embodiments, the amino acid sequence is selected from the group consisting of the amino acid sequence of C. jejuni 81116, HB-95-29, INP-59 EF-Tu, INP44 and C. coli D3088 EF-Tu (SEQ ID NO:7); the amino acid sequence of C. jejuni 81-176 ortholog of Cj0069 (SEQ ID NO:9); the amino acid sequence of C. jejuni 81-176 ortholog of Cj0561c (SEQ ID NO:11); the amino acid sequence of C. jejuni Arylsulfatase (SEQ ID NO:12); the amino acid sequence of M. tuberculosis Rv2794c (SEQ ID NO:13); the amino acid sequence of D. radiodurans DRC0015 (SEQ ID NO:14); and orthologs and homologs thereof.

The polypeptides of the invention or their representative fragments entering into the immunogenic compositions according to the invention may be selected by techniques known to persons skilled in the art, such as for example on the capacity of the said polypeptides to stimulate T cells, which results, for example, in their proliferation or the secretion of interleukins, and which leads to the production of antibodies directed against the said polypeptides.

Compositions, suitable to be used as immunogenic compositions, may be prepared from Campylobacter outer membrane proteins, analogs and fragments thereof, peptides and nucleic acid molecules encoding such Campylobacter outer membrane proteins, fragments and analogs thereof and peptides as disclosed herein. The immunogenic composition elicits an immune response that produces antibodies, including antibodies directed to the Campylobacter outer membrane protein, and preferably elicits production of antibodies that are opsonizing or bactericidal.

The nucleic acid molecules encoding the Campylobacter outer membrane protein, fragments or analogs thereof, of the present invention may also be used directly for immunization by administration of the nucleic acid molecule (including DNA molecules) directly, for example by injection for genetic immunization or by constructing a live vector such as Salmonella, BCG, adenovirus, poxvirus, vaccinia or poliovirus.

The Campylobacter outer membrane proteins, analogs and fragments thereof and/or peptides of the present invention are useful as immunogens, as antigens in immunoassays including enzyme-linked immunosorbent assays (ELISA), RIAs and other non-enzyme linked antibody binding assays or procedures known in the art for the detection of anti-bacterial, Campylobacter outer membrane proteins and/or peptide antibodies.

Because of their high specificity, the monoclonal antibodies may be a useful reagent for the detection of C. jejuni and/or C. coli in foods, clinical specimens, or in situ localization of the bacteria. The antibodies of the present invention may be used further to monitor environmental and/or waste water or various facilities for C. jejuni and/or C. coli contamination. The testing procedure would include, for example, enzyme-linked immunoassays (ELISAs), immunomagnetic capture, radioimmune assays, biosensor assays and other immunoassays including, but not limited to microscopic methods.

The monoclonal antibodies of the present invention may be used singly or in combination, such as in a cocktail mixture, to detect specific Campylobacter species, as neutralizing antibodies, or in a composition comprising one or more antibodies to be used in administering passive immunity to humans, livestock, poultry, or other animals.

The antibodies may be used further in side-by-side assays to determine whether the samples react only with the antibodies specific for both C. jejuni and C. coli or whether they react only with the antibodies that are specific for C. jejuni alone.

Another object of the invention is to provide an immunogen comprising at least a portion of an outer membrane protein that is present and conserved in multiple strains of C. jejuni used as a immunogenic composition which induces high levels of specific antibodies directed against C. jejuni and which protects against C. jejuni infection in humans, livestock, poultry, or other animals.

An additional object of the invention is to provide an immunogen comprising at least a portion of an outer membrane protein that is present and conserved in multiple strains of C. jejuni and C. coli, wherein when the immunogen is used as a immunogenic composition, induces high levels of specific antibodies directed against both C. jejuni and C. coli and which protects against C. jejuni and C. coli infection in humans, livestock, poultry, or other animals.

As used herein, the terms “polynucleotide,” “oligonucleotide,” “nucleic acid” and “nucleic acid molecule” include a polymeric form of nucleotides of any length, either ribonucleotides or deoxyribonucleotides. This term refers only to the primary structure of the molecule; thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing non-nucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine and others can also be used for antisense, dsRNA and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

An “isolated” nucleic acid molecule is one that is substantially separated from other nucleic acid molecules that are present in the natural source of the nucleic acid (i.e., sequences encoding other polypeptides). Preferably, an “isolated” nucleic acid is free of some of the sequences that naturally flank the nucleic acid (i.e., sequences located at the 5′ and 3′ ends of the nucleic acid) in its naturally occurring replicon. For example, a cloned nucleic acid is considered isolated. A nucleic acid is also considered isolated if it has been altered by human intervention, or placed in a locus or location that is not its natural site, or if it is introduced into a cell by transfection. Moreover, an “isolated” nucleic acid molecule can be free from some of the other cellular material with which it is naturally associated, or culture medium when produced by recombinant techniques, or chemical precursors or other chemicals when chemically synthesized.

Specifically excluded from the definition of “isolated nucleic acids” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified nucleic acid makes up less than 5% of the number of nucleic acid inserts in the vector molecules. Further specifically excluded are whole cell genomic DNA or whole cell RNA preparations (including whole cell preparations that are mechanically sheared or enzymatically digested). Even further specifically excluded are the whole cell preparations found as either an in vitro preparation or as a heterogeneous mixture separated by electrophoresis wherein the nucleic acid of the invention has not further been separated from the heterologous nucleic acids in the electrophoresis medium (e.g., further separating by excising a single band from a heterogeneous band population in an agarose gel or nylon blot).

In one preferred embodiment, an isolated nucleic acid encoding a Campylobacter outer membrane protein can be chimeric or fusion polynucleotides. As used herein, a “chimeric polynucleotide” or “fusion polynucleotide” comprises a nucleic acid encoding a Campylobacter outer membrane peptide operably linked to a second nucleic acid sequence. Preferably, the second nucleic acid sequence is not a Campylobacter outer membrane, and has both a different polynucleotide sequence and encodes a protein having a different function than a nucleic acid encoding a Campylobacter outer membrane peptide. Within the fusion polynucleotide, the term “operably linked” is intended to indicate that the nucleic acid encoding a Campylobacter outer membrane peptide and the second nucleic acid sequence, respectively, are fused to each other so that both sequences fulfill the proposed function attributed to the sequence used. The second nucleic acid sequence can be fused to the N-terminus or C-terminus of the nucleic acid encoding a Campylobacter outer membrane peptide.

Procedures for introducing a nucleic acid into a cell are well known to those of ordinary skill in the art, and include, without limitation, transfection, transformation or transduction, electroporation, particle bombardment, agroinfection, and the like. In certain embodiments, the nucleic acid is incorporated into a vector or expression cassette that is then introduced into the cell. Other suitable methods for introducing nucleic acids into host cells can be found in Sambrook, et al., Molecular Cloning: A Laboratory Manual. 2nd Ed., Cold Spring Harbor Laboratory, Cold Spring Harbor Laboratory Press, Cold Spring Harbor, N.Y., 1989, and other laboratory manuals such as Methods in Molecular Biology, 1995, Vol. 44, Agrobacterium protocols, Ed: Gartland and Davey, Humana Press, Totowa, N.J.

As used herein, the term polypeptide refers to a chain of at least four amino acids joined by peptide bonds. The chain may be linear, branched, circular or combinations thereof. The terms “peptide,” “polypeptide,” and “protein” are used interchangeably herein. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

In certain embodiments, the invention encompasses a polypeptide as defined in any one of (a) SEQ ID NO:7, SEQ ID NO:9, SEQ ID NO:11, SEQ ID NO:12, SEQ ID NO:13, or SEQ ID NO:14; (b) a 50 amino acid fragment of any of (a); and (c) an ortholog or homolog of any of a).

The invention also provides chimeric or fusion polypeptides. As used herein, an “chimeric polypeptide” or “fusion polypeptide” comprises a Campylobacter outer membrane polypeptide operatively linked to a second polypeptide, also termed a fusion protein partner. Preferably the second polypeptide has an amino acid sequence that is not substantially identical to a Campylobacter outer membrane polypeptide, e.g., a polypeptide that is not expressed at substantial levels on the outer membrane of a Campylobacter species. As used herein with respect to the fusion polypeptide, the term “operatively linked” is intended to indicate that the two polypeptides are fused to each other so that both sequences fulfill the proposed function attributed to the sequence used. The second polypeptide can be fused to the N-terminus or C-terminus of the Campylobacter outer membrane polypeptide.

A suitable fusion protein partner consists of a protein that will either enhance or at least not diminish the recombinant expression of the Campylobacter fusion protein product when the two are in genetic association. Still further, a suitable fusion partner will facilitate the purification of the chimeric Campylobacter fusion protein. A representative list of suitable fusion protein partners includes maltose binding protein, poly-histidine segments capable of binding metal ions, inteine, antigens to which antibodies bind, S-Tag, glutathione-S-transferase, thioredoxin, beta-galactosidase, nonapeptide epitope tag from influenza hemagglutinin, a 11-amino acid epitope tag from vesicular stomatitis virus, a 12-amino acid epitope from the heavy chain of human Protein C, green fluorescent protein, cholera holo toxin or its B subunit, E. coli heat-labile holotoxin or its B subunit, CTA1-DD, streptavidin and dihydrofolate reductase.

To determine the percent sequence identity of two amino acid sequences, the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence, then the molecules are identical at that position. The same type of comparison can be made between two nucleic acid sequences.

The percent sequence identity between the two sequences is a function of the number of identical positions shared by the sequences (i.e., percent sequence identity=numbers of identical positions/total numbers of positions×100). The preferable length of sequence comparison for nucleic acids is at least 75 nucleotides, more preferably at least 100 nucleotides and most preferably the entire length of the coding region.

For the purposes of the invention, the percent sequence identity between two polynucleotide or polypeptide sequences is determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two polynucleotides. A gap opening penalty of 10 and a gap extension penalty of 0.1 are used for determining the percent identity of two polypeptides. All other parameters are set at the default settings. It is to be understood that for the purposes of determining sequence identity when comparing a DNA sequence to an RNA sequence, a thymidine nucleotide is equivalent to a uracil nucleotide.

In another aspect, the invention provides an expression vector, or host cell comprising a polynucleotide that hybridizes to the polynucleotide encoding a Campylobacter outer membrane protein or fragment thereof under stringent conditions, wherein the hybridizing sequence is operably linked to a regulatable promoter. More particularly, a hybridizing sequence is at least 15 nucleotides in length and hybridizes under stringent conditions to the nucleic acid molecule encoding a Campylobacter outer membrane protein or fragment thereof. In other embodiments, the nucleic acid is at least 30, 50, 100, 250 or more nucleotides in length.

As used herein with regard to hybridization for DNA to DNA blot, the term “stringent conditions” refers to hybridization overnight at 60° C. in 10× Denhart's solution, 6×SSC, 0.5% SDS and 100 μg/ml denatured salmon sperm DNA. Blots are washed sequentially at 62° C. for 30 minutes each time in 3×SSC/0.1% SDS, followed by 1×SSC/0.1% SDS and finally 0.1×SSC/0.1% SDS. As also used herein, “highly stringent conditions” refers to hybridization overnight at 65° C. in 10× Denhart's solution, 6×SSC, 0.5% SDS and 100 g/ml denatured salmon sperm DNA. Blots are washed sequentially at 65° C. for 30 minutes each time in 3×SSC/0.1% SDS, followed by 1×SSC/0.1% SDS and finally 0.1×SSC/0.1% SDS. Methods for nucleic acid hybridizations are described in Meinkoth and Wahl, 1984 Anal. Biochem. 138:267-284; Current Protocols in Molecular Biology, Chapter 2, Ausubel et al., Eds., Greene Publishing and Wiley-Interscience, New York, 1995; and Tijssen, Laboratory Techniques in Biochemistry and Molecular Biology: Hybridization with Nucleic Acid Probes, Part I, Chapter 2, Elsevier, New York, 1993. Preferably, an isolated nucleic acid molecule of the invention that hybridizes under stringent or highly stringent conditions to a sequence encoding a Campylobacter outer membrane protein or fragment thereof corresponds to a naturally occurring nucleic acid molecule. As used herein, a “naturally occurring” nucleic acid molecule refers to an RNA or DNA molecule having a nucleotide sequence that occurs in nature (e.g., encodes a natural polypeptide).

Using the above-described methods, and others known to those of skill in the art, one of ordinary skill in the art can isolate homologs of the polypeptides encoding a Campylobacter outer membrane protein. One subset of these homologs is allelic variants. As used herein, the term “allelic variant” refers to a nucleotide sequence containing polymorphisms that lead to changes in the amino acid sequences of a Campylobacter outer membrane protein and that exist within a natural population (e.g., a plant species or variety). Such natural allelic variations can typically result in 1-20% variance in a nucleic acid encoding a Campylobacter outer membrane protein. Allelic variants are intended to be within the scope of the invention.

Moreover, nucleic acid molecules encoding a Campylobacter outer membrane protein from the same or other species such as analogs, orthologs and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov et al., 1997 Science 278(5338):631-637).

In addition to naturally-occurring variants of a gene encoding a Campylobacter outer membrane protein that may exist in the population, the skilled artisan will further appreciate that changes can be introduced by mutation into the nucleotide sequence, thereby leading to changes in the amino acid sequence of the encoded Campylobacter outer membrane polypeptide, without altering the functional activity of the polypeptide. For example, nucleotide substitutions leading to amino acid substitutions at “non-essential” amino acid residues can be made in the sequence. A “non-essential” amino acid residue is a residue that can be altered from the wild-type sequence of a polypeptide without altering the activity of said polypeptide, whereas an “essential” amino acid residue is required for polypeptide activity. Other amino acid residues, however, may not be essential for activity and thus are likely to be amenable to alteration without altering the activity of the outer membrane protein.

Accordingly, another aspect of the invention pertains to Campylobacter outer membrane polypeptides that contain changes in amino acid residues that are not essential for their activity. An isolated nucleic acid molecule encoding a polypeptide having sequence identity with a polypeptide sequence of a Campylobacter outer membrane protein can be created by introducing one or more nucleotide substitutions, additions or deletions into a nucleotide sequence encoding a Campylobacter outer membrane polypeptide, such that one or more amino acid substitutions, additions or deletions are introduced into the encoded polypeptide. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Thus, a predicted nonessential amino acid residue in a Campylobacter outer membrane polypeptide is preferably replaced with another amino acid residue from the same side chain family. Alternatively, in another embodiment, mutations can be introduced randomly along all or part of a Campylobacter outer membrane polypeptide coding sequence, such as by saturation mutagenesis, and the resultant mutants can be screened for an activity as described herein to identify mutants that retain activity. Following mutagenesis of the sequence, the encoded polypeptide can be expressed recombinantly and the activity of the polypeptide can be determined by analyzing a cell expressing the polypeptide.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990) or see: Gruber & Crosby, in: Methods in Plant Molecular Biology and Biotechnology, eds. Glick & Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

Another aspect to the invention is the generation and the purification or isolation of antibodies that specifically bind the outer membrane proteins or immunogenic fragments thereof. As used herein, the term “antibody” is intended to include immunoglobulin molecules and immunologically active portions of immunoglobulin molecules, i.e., molecules that contain an antigen binding site that specifically binds (immunoreacts with) an antigen, such as Fab and F(ab′)₂ fragments. As used herein, the term “antibody” includes polyclonal and monoclonal antibodies, and variants such as single-chain (recombinant) antibodies, “humanized” chimeric antibodies, and immunologically active fragments of antibodies. For the purposes of this invention, a “chimeric” monoclonal antibody is a murine monoclonal antibody comprising constant region fragments (Fc) from a different animal. For the purposes of this invention, a “humanized” monoclonal antibody is a murine monoclonal antibody in which human protein sequences have been substituted for all the murine protein sequences except for the murine complementarity determining regions (CDR) of both the light and heavy chains. Standard techniques for the generation and isolation of antibodies are well-known and commonly employed by those of skill in the art. A number of standard techniques are described in Kohler & Milstein, 1975, Nature 256:495-497; Kozbor et al., 1983, Immunol Today 4:72; Cole et al., 1985, Monoclonal Antibodies and Cancer Therapy, Alan R. Liss, Inc., pp. 77-96; Kenneth, in Monoclonal Antibodies: A New Dimension In Biological Analyses, Plenum Publishing Corp., New York, N.Y. (1980); Lemer, 1981, Yale J. Biol. Med., 54:387-402; M. L. Gefter et al., 1977, Somatic Cell Genet., 3:231-36; and Galfre et al., 1977, Nature 266:55052.

The invention further encompasses methods of using the antibodies for therapeutic, diagnostic and experimental purposes. The antibodies described and claimed herein are useful for isolating the proteins to which the monoclonal antibodies bind. The antibodies are also valuable in new and useful methods, including, but not limited to, methods for inhibiting the immune response in an animal, and for determining whether an animal has been infected with Campylobacter.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods that occur to the skilled artisan are intended to fall within the scope of the present invention.

### EXAMPLES

### Example 1

Proteomic Analysis of C. jejuni Outer Membrane Proteins

C. jejuni outer membranes were purified using standard methodology (Hickey et al., 1999 Infect Immun., 67:88-93; Thompson & Sparling, 1993 Infect Immun., 61:2906-2911). Briefly, C. jejuni strains 81-176 and NCTC 11168 were grown to mid-log phase in Brucella broth at 37° C., and harvested into 50 mL polypropylene tubes containing 4 μL chloramphenicol (32 mg/mL) per 1 mL of culture volume at 4° C. Cells were pelleted at 4000 rpm for 20 minutes at 4° C. The supernatant was discarded, and the pellet was resuspended in 1 mL of 10 mM HEPES, pH 7.4 and transferred to 1.5 mL microfuge tube. The cells were lysed by sonication (Sonic Dismembrator 60, Fisher Scientific) at power setting 7 using 10 second bursts followed by 20 seconds in ice for approximately 5 cycles, and residual unlysed cells were pelleted by low speed centrifugation for 5 minutes at 14,000 rpm. The supernatant was transferred to an ultracentrifuge tube and the pellet was discarded. Total membranes were recovered from the cell lysate supernatant by ultracentrifugation at 40,000 rpm and 4° C. for 1 hour. After the supernatant was discarded, the pellet was resuspended in 10 mM HEPES, pH 7.4 containing 5% Sarkosyl, and again centrifuged at 40,000 rpm and 4° C. for 1 hour. This step was repeated. The resulting pellet was resuspended in 10 mM HEPES, pH 7.4, centrifuged at 40,000 rpm and 4° C. for 1 hour, and the supernatant discarded. This step was repeated. All but 200 μL of the supernatant was discarded. The pellet was resuspended in the remaining supernatant. The outer membrane protein suspension was kept at −70° C. for long-term storage or sampled for BCA assay to determine protein concentration for proteomics labeling.

C. jejuni outer membrane proteins were visualized in 2-D gels both by staining the proteins with Sypro Ruby, and by fluorescently labeling the proteins directly with Cy5 dye. C. jejuni outer membrane proteins were first subjected to isoelectric focusing (IEF) using the Ettan IGPhor IEF apparatus (Amersham Biotech) using Immobiline IEF strips (linear pI range 3-10).

For isoelectric focusing in the first dimension, the protein concentration in the lysate sample(s) was determined by BCA assay (Pierce, Rockford, Ill.). 50 ug of each sample was aliquoted into a microfuge tube, one sample per tube. 1 μL of 1:10 diluted Cy dye was added to each sample, using a different dye type per different sample. The samples were mixed well and centrifuged briefly (3000 rpm for 15 seconds). The tubes were placed on ice and covered for 30 minutes. 1 μL of 10 mM lysine was added to each sample, mixed as before, and returned covered to ice for 10 minutes. The samples were combined and the total volume determined. An equal volume of 2× sample buffer was added and placed on ice for 15 minutes. The volume was brought to 250 μL with rehydration buffer. The samples were laid out in a continuous line between the electrodes of the focusing coffin. The IPG drystrip was placed on the sample gel side down. The strip was overlaid with 1-1.5 mL of dry strip cover fluid (mineral oil). The coffin was placed in the isoelectric focuser at the appropriate position as determined by strip length and focus according to the following parameters: S1 100V 1:00 hrs; S2 500V 1000Vhr; S3 1000V 2000 Vhr; S4 2000V 4000 Vhr; S5 4000V 8000 Vhr; S6 8000V 8:00 hrs; and S7 500V 24:00 hrs. Upon completion of the focus, the strip was removed from the coffin and placed in an empty Petri dish for storage for up to several weeks at −20° C.

After IEF, strips were laid on a 7.5% SDS-PAGE gel for second dimension electrophoresis using techniques well known to those of skill in the art.

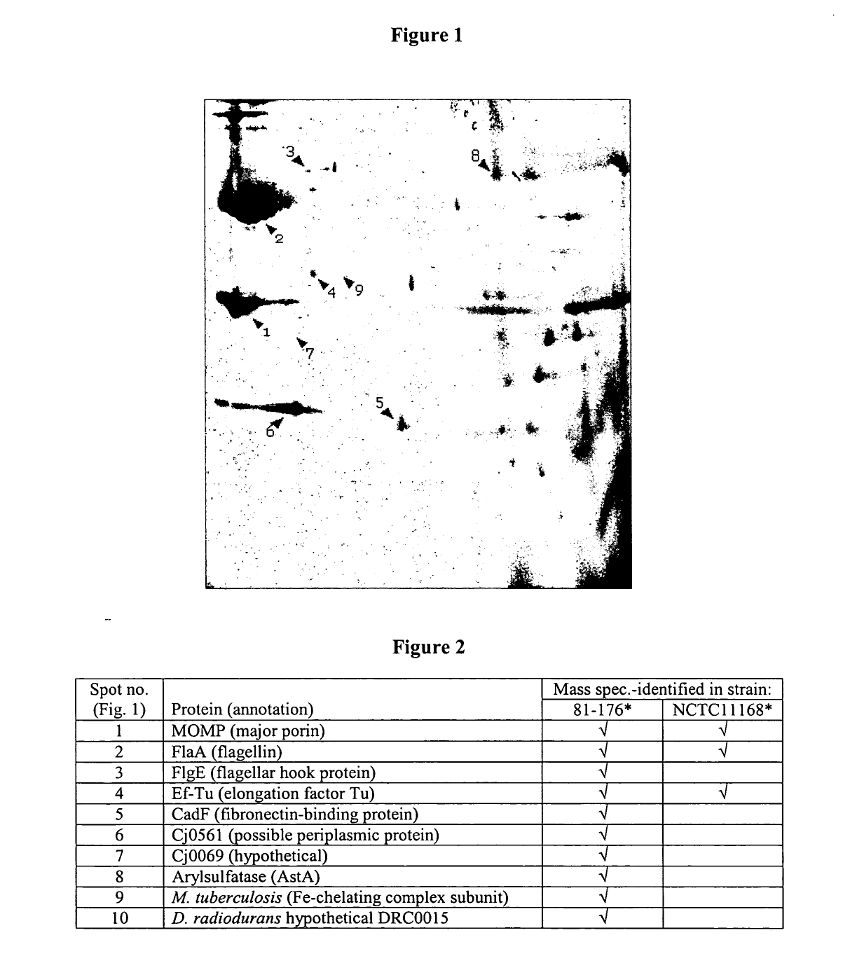

Following second dimension separation, the 2-D gel was scanned using an Amersham Biotech Typhoon scanner to visualize the protein spots. A C. jejuni 81-176 outer membrane protein profile (labeled with Cy5) is shown in FIG. 1. A spot map of the outer membrane proteins was generated, and the coordinates of the spots were exported to an Ettan robotic spot picker, which cored the spots of interest and arrayed them in a 96-well microtiter dish. Proteins were extracted from the acrylamide plugs and digested with trypsin, using an Ettan robotic digester. Tryptic protein digests were then subjected to Matrix Assisted Laser Desorption Ionization—Time of Flight (MALDI-ToF) mass spectrometry using both Ettan (Amersham Biotech) and Voyager (Applied Biosystems) MALDI-ToF spectrometers to generate a peptide fingerprint for each isolated protein. The tryptic fingerprints were then compared to databases of similarly digested proteins from NCBI and SwissProt, including the proteins predicted by the C. jejuni NCTC11168 genome sequence. Results of database searches include the alignments of peptide matches, amount of coverage of the total protein, and the statistical significance of the matches.

### Example 2

Identification of Outer Membrane Proteins

Using the methods as described in Example 1, preliminary mass spectrometry analysis of C. jejuni outer membrane proteins has allowed for the identification of some of the known, major proteins found in the C. jejuni outer membrane. Some of these are already known to be variable at the level of primary amino acid sequence, and some are not found in all C. jejuni strains. Interestingly, some of the proteins found in the outer membrane, such as EF-Tu, were known to be present in C. jejuni, but were not previously known to be outer membrane proteins, and thereby, may be novel subunit immunogenic composition candidates. Others proteins identified had not previously been characterized in C. jejuni, but had been characterized in other organisms such as M. tuberculosis.

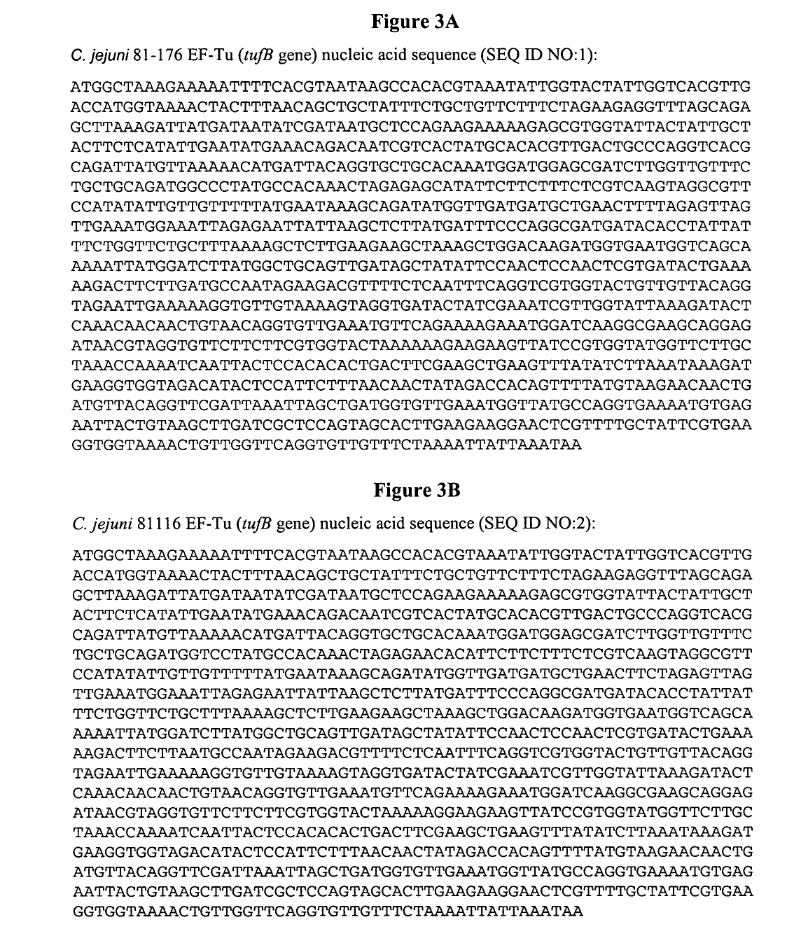

The preliminary screen identified 10 outer membrane proteins, including major outer membrane protein, Flagellin A, flagellar hook protein (FlgE), Elongation factor Tu, Campylobacter fibronectin binding protein (CadF), Cj0561c, Cj0069, Arylsulfatase, and orthologs of M. tuberculosis Rv2794c, and D. radiodurans hypothetical protein DRC0015. These proteins are identified in FIG. 2.

Major outer membrane protein (MOMP) is the major porin of C. jejuni (Bollaet et al., J Bacteriol., 177:4266-4271; Huyer et al., 1986 FEMS Microbiol Lett., 37:247-250), and can be visualized on the Campylobacter cell surface by electron microscopy (Amako et al., 1997 Microbiol Immunol., 41:855-859; Amako et al., 1996 Microbiol Immunol., 40:749-754). MOMP may also have alternate functions, such as adherence to host cells (Moser et al., 1997 FEMS Microbiol Lett., 157:233-238; Schroder & Moser, 1997 FEMS Microbiol Lett., 150:141-147), antibiotic resistance (Page et al., 1989 Antimicrob Agents Chemother., 33:297-303), and possibly cytotoxicity (Bacon et al., 1999 J Med Microbiol., 48:139-14). Although MOMP is highly immunogenic in humans (Blaser et al., 1984 Infect Immun., 43:986-993; Cawthraw et al., 2002 Clin Exp Immunol., 130:101-106; Nachamkin & Hart, 1985 J Clin Microbiol., 21:33-38; Panigrahi et al., 1992 Infect Immun., 60:4938-4944), it is also variable among strains (Zhang et al., 2000 Infect Immun., 68:5679-5689).

Flagellin A (FlaA) is the major subunit (flagellin) of C. jejuni flagella, a major virulence factor that protrudes from the C. jejuni cell surface (Guerry, 2000 p. 405-421, In Nachamkin & Blaser (ed.), Campylobacter, 2nd ed. ASM Press, Washington, D.C.). As described herein, FlaA is a well-characterized and abundant OMP constituent that is widely conserved among C. jejuni strains.

FlgE is the flagellar hook protein, a component of the flagellar apparatus that is located in the C. jejuni outer membrane and is surface exposed (Lüneberg et al., 1998 J Bacteriol., 180:3711-3714). As an essential component of highly conserved flagella, FlgE was found in each of a limited number of C. jejuni strains, although the sizes of FlgE varied considerably. The surface exposed regions of FlgE are hypervariable (Lüneberg et al., 1998 J Bacteriol., 180:3711-3714).

Elongation factor Tu (EF-Tu) is a cytoplasmic protein that is a critical component of the bacterial translation machinery, mediating the binding of aminoacyl tRNA to the ribosomal A site. However, recently it has been shown to have other localizations and functions as well. EF-Tu has been reported on the cell surface of M. pneumoniae, where it serves as a fibronectin binding protein (35) and may play a role in adherence to host cells and virulence. Interestingly, EF-Tu is one of the most highly immunogenic proteins of H. pylori (88).

CadF mediates the binding of C. jejuni and extracellular matrix fibronectin (Konkel et al., 1997 Mol Microbiol., 24:953-963). Consequently, CadF is considered as a C. jejuni adherence factor. CadF is immunogenic in human convalescent serum, and polyclonal rabbit serum detected CadF in all tested C. jejuni strains (Konkel et al., 1997 Mol Microbiol., 24:953-963).

Cj0561c was identified. This protein was annotated in the NCTC11168 sequence as a “possible periplasmic protein.” However, it is possible from sequence analysis that this annotation was in error, and that this protein is fact an outer membrane protein. The amino acid sequence possesses a Gram-negative signal peptide (Nielsen et al., 1997 Protein Eng., 10:1-6), has a predicted transmembrane domain at amino acids 193-214, and ends in the motif YKF, a signature of outer membrane proteins (Cover et al., 1994 J Biol Chem., 269:10566-10573; Farizo et al., 2002 Infect Immun., 70:1193-1201; Jansen et al., 2000 Eur J Biochem., 267:3792-3800; Struyvé et al., 1991 J Mol Biol., 218:141-148).

Cj0069 was identified. Cj0069 was predicted as a hypothetical protein by the NCTC11168 genome sequence, although nothing further is known about this protein. It is a 39 kDa protein with similarity only to hypothetical proteins of Corynebacterium and Bradyrhizobium (GenBank accession numbers NP_{—}737331 and NP_{—}774709, respectively). Cj0069 lacks a signal peptide and transmembrane domains, but was determined herein to be present in a C. jejuni 81-176 outer membrane fraction.

Arylsulfatase (AstA) was identified. C. jejuni arylsulfatase from strain 81-176 was first reported by Yao and Guerry (1996 J Bacteriol., 178:3335-3338), and is a degradative enzyme purported to be a C. jejuni virulence factor. Although little else is known about the C. jejuni protein, E. coli K1 arylsulfatase is involved in cell invasion (Hoffman et al., 2000 Infect Immun., 68:5062-5067) and in E. coli K12 is activated by OxyR as part of an oxidative stress regulon (Mukhopadhyay & Schellhorn, 1997 J Bacteriol., 179:330-338). No ortholog of AstA was found in the NCTC11168 sequence (Parkhill et al., 2000 Nature, 403:665-668), substantiating that this protein exhibits interstrain variability Guerry (Yao & Guerry, 1996 J Bacteriol., 178:3335-3338). The C. jejuni AstA protein has a signal peptide (Nielsen et al., 1997 Protein Eng., 10:1-6), suggesting periplasmic or outer membrane localization.

M. tuberculosis Rv2794c was identified in the screen. Although annotated in the published sequence (Cole et al., 1998 Nature, 393:537-544) as a “hypothetical protein,” TIGR has designated Rv2794c as a “putative iron-chelating complex subunit” (TIGR, 2003, [http://www.tigr.org/tigr-scripts/CMR2/GenomePage3.spl?database=ntmt02]). A signal peptide is predicted (Nielsen et al., 1997 Protein Eng., 10:1-6), indicating extracytoplasmic localization.

D. radiodurans hypothetical protein DRC0015 was identified. DRC0015 (White et al., 1999 Science, 286:1571-1577) has a signal peptide (Nielsen et al., 1997 Protein Eng., 10: 1-6), but is a C. jejuni ortholog that has not been previously noted.

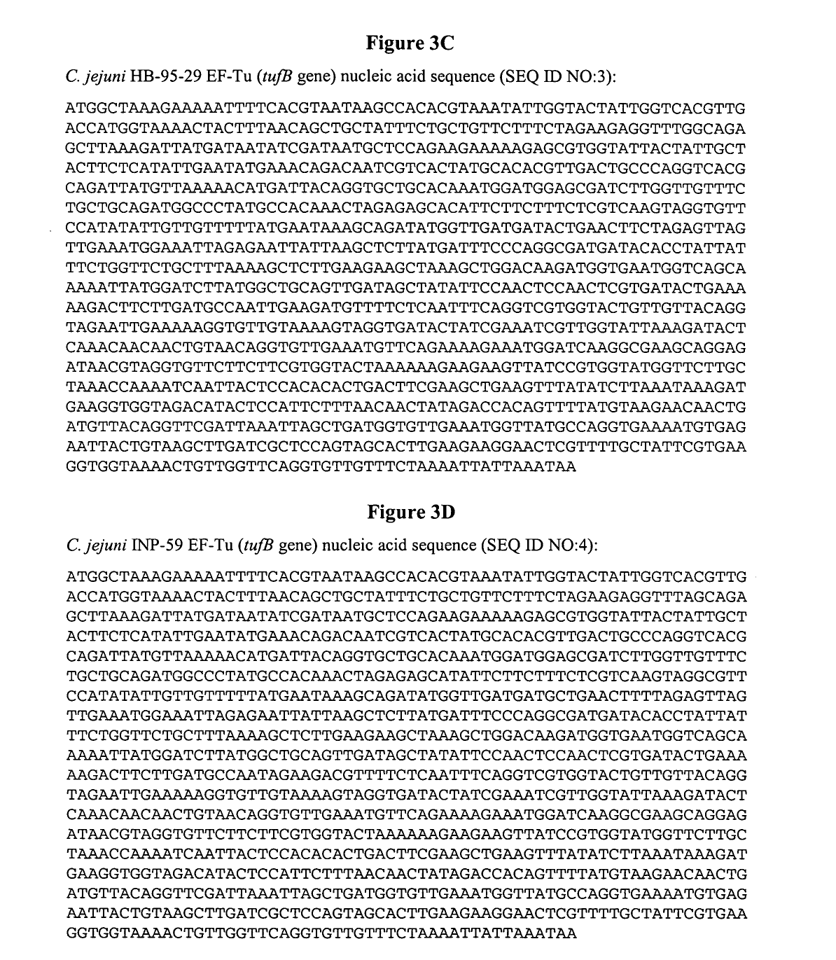

The nucleotide sequences of C. jejuni 81-176 EF-Tu (tufB gene; SEQ ID NO:1); C. jejuni 81116 EF-Tu (tufB gene; SEQ ID NO:2); C. jejuni HB-95-29 EF-Tu (tufB gene; SEQ ID NO:3); C. jejuni INP-59 EF-Tu (tufB gene; SEQ ID NO:4); C. jejuni INP44 EF-Tu (tufB gene; SEQ ID NO:5); and C. coli D3088 EF-Tu (tufB gene; SEQ ID NO:6) are provided in FIGS. 3A-F. The amino acid sequence of C. jejuni 81116, HB-95-29, INP-59 EF-Tu, INP44 and C. coli D3088 EF-Tu (SEQ ID NO:7) is provided in FIG. 3G.

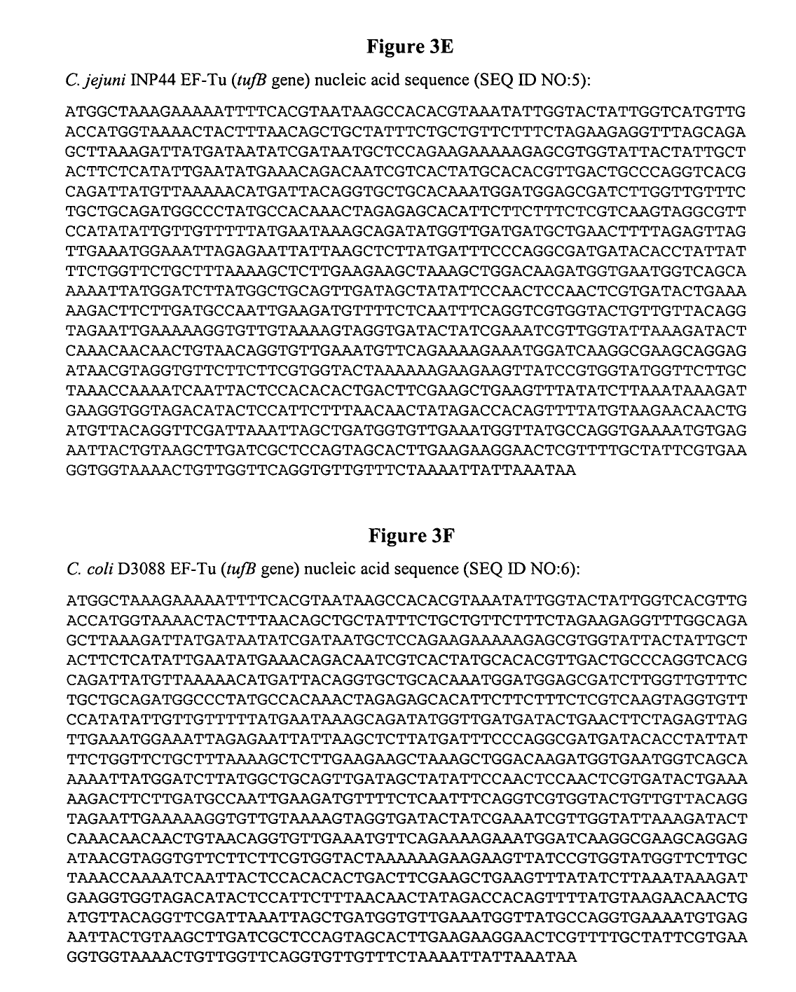

The nucleotide sequence of C. jejuni 81-176 ortholog of Cj0069 (SEQ ID NO:8); and the amino acid sequence of C. jejuni 81-176 ortholog of Cj0069 (SEQ ID NO:9) are provided in FIGS. 4A-B, respectively.

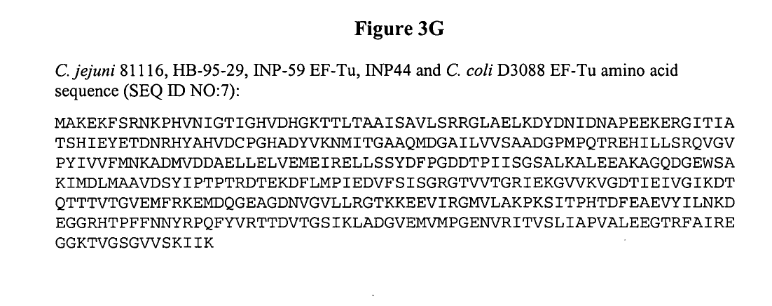

The nucleotide sequence of C. jejuni 81-176 ortholog of Cj0561C (SEQ ID NO:10); and the amino acid sequence of C. jejuni 81-176 ortholog of Cj0561C (SEQ ID NO:11) are provided in FIGS. 5A-B, respectively.

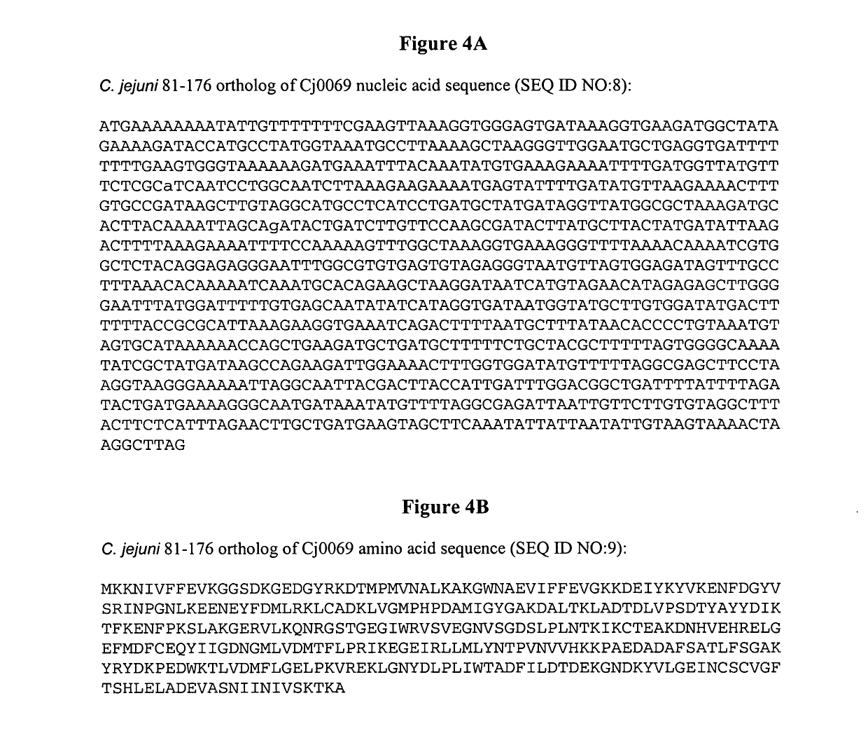

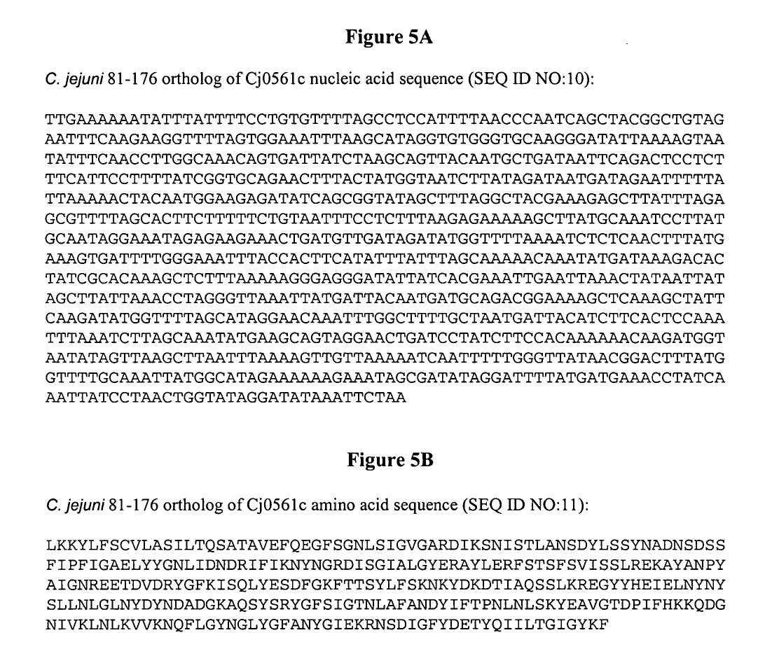

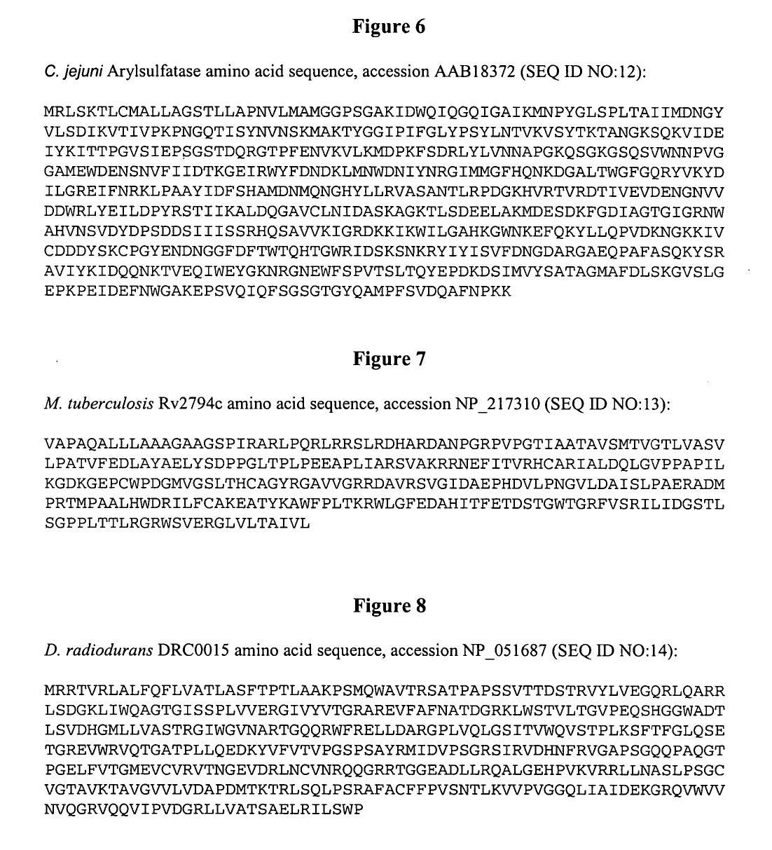

The amino acid sequences of C. jejuni Arylsulfatase (SEQ ID NO:12); M. tuberculosis Rv2794c (SEQ ID NO:13); and D. radiodurans DRC0015 (SEQ ID NO:14) are provided in FIGS. 6, 7, and 8, respectively.

### Example 3

Identification of Outer Membrane Proteins from 7 Different Campylobacter Strains

The outer membrane proteins of 7 different Campylobacter strains are identified to find highly conserved proteins as the basis for a subunit immunogenic composition. Two approaches are used. First, proteomics/mass spectrometry is used to directly identify the proteins that constitute the outer membranes of these 7 Campylobacter strains. Second, the immunogenicity of the outer membrane proteins in humans is identified by immunoblotting 2-D protein gels with convalescent human serum.

The identities of the outer membrane proteins of interest are confirmed by creating specific mutants lacking the outer membrane proteins. Representative outer membrane protein-encoding genes are sequenced from each of the 7 strains to determine the degree to which these genes (and the encoded outer membrane proteins) are conserved.

The seven Campylobacter strains tested are C. jejuni 81-176, NCTC11168, 81116, HB95-29, INP59, INP44, and C. coli D3088.

The proteomics experiments are performed as described in Example 1. In addition, the C. jejuni strains are grown in the presence and absence of Desferal, an iron chelator known to induce the expression of iron-repressible proteins (many of which are typically outer membrane proteins) in many bacteria, including C. jejuni (Dyer et al., 1988 Infect Immun., 56:977-983; Thompson et al., 1993 J Bacteriol., 175:811-818; van Vliet et al., 1999 J Bacteriol., 181:6371-6376; van Vliet et al., 2000 FEMS Microbiol Lett., 188:115-118; van Vliet et al., 1998 J Bacteriol., 180:5291-5298). This may allow the identification of outer membrane proteins that are repressed under normal iron-replete growth, but that become highly expressed under conditions of iron-depletion, such as found in the human body.

The outer membrane proteins of each of the 7 Campylobacter strains are subjected to isoelectric focusing (IEF) on an Amersham IPGPhor apparatus using Immobiline DryStrip IEF strips. The optimal separation conditions for resolving different proteins are determined by using IEF strips with differing pI ranges (i.e. 3-10, 3-7, and 6-11) and differing concentrations of acrylamide for second dimension separation.

### Example 4

Characterization of Immunogenic Outer Membrane Proteins

2-D gels are performed on outer membrane proteins from the 7 Campylobacter strains as described above. The separated outer membrane proteins are then transferred to PVDF membranes as described previously (Bumann et al., 2002 Infect Immun., 70:6494-6498; Jungblut et al., 2000 Mol Microbiol., 36:710-725). These membranes are probed with convalescent human serum from patients with C. jejuni infection. Pools of convalescent patient sera (each pool consisting of sera from 20 patients with no prior history of Campylobacter infection) are obtained from patients presenting with C. jejuni diarrhea. Pooled serum from volunteers with no history of Campylobacter infection serves as a control for non-specific binding of patient serum to C. jejuni proteins. This is compared to the immunoreactivity of C. jejuni outer membrane proteins with convalescent serum containing antibodies against C. jejuni. This identifies those outer membrane proteins that are the most immunogenic during infection. These protein spots are picked and identified by mass spectrometry.

If a database match from a protein of reasonable abundance and high quality mass spectrum cannot be made, two de novo sequencing methods are attempted. The first uses the Applied Biosystems Q-Star (quadrupole-time-of-flight) mass spectrometer that allows de novo protein sequence to be derived from trypsin-digested peptides of a protein separated on an accompanying Agilent HPLC. Additionally, the Amersham chemically assisted fragmentation (CAF)-MALDI mass spectrometry is attempted. CAF-MALDI allows the generation of protein sequence from peptides that are chemically fragmented in a sequence-specific manner. Either techniques yields the primary amino acid sequence of proteins of interest, which are searched against protein databases using BLASTP. This may allow identification of proteins even if they are glycosylated, have divergent sequence, or have homologs not in NCTC11168 but in other closely related organisms such as H. pylori. In the absence of a protein database match, the experimentally determined peptide sequences allow the cloning of the gene of interest from the strain in which it was identified and the novel gene is characterized.

### Example 5

Characterization of Genes Encoding Immunogenic Outer Membrane Proteins

The degree of conservation of the outer membrane proteins is determined at the level of primary amino acid sequence among the different strains. Consequently, PCR primers are designed based on the published nucleotide sequences of the genes encoding these proteins. In the majority of cases this is likely to be the NCTC11168 genome sequence (Parkhill et al., 2000 Nature, 403:665-668), although if the protein of interest is not in the published genome sequence PCR primers are designed based on the available corresponding GenBank sequence.

PCR is performed using these primers and a high-fidelity Taq polymerase (PfuTurbo; Stratagene, La Jolla, Calif.), to amplify the genes of interest from each of the 7 Campylobacter strains. The DNA sequences of the PCR products are determined, predicting the deduced amino acid sequences. The amino acid sequences are used to determine the degree to which the predicted proteins are conserved among the initial 7 Campylobacter strains. Proteins that are moderately to highly divergent between strains will have a lesser possibility of generating a protective immune response to heterologous strains, and are not pursued further. In contrast, a high degree of amino acid identity between strains indicates that the proteins are highly conserved and therefore are candidates for further study.

Isogenic mutants lacking the specific outer membrane proteins of primary interest are constructed. These are made by simple insertion of an antibiotic resistance cassette (in the case of monocistronic genes, or those at the 3′ end of operons) or the engineering of non-polar chromosomal mutations (for genes organized in operons). In the case of simple insertions, a portion of the gene of interest is amplified using PCR and gene specific primers designed from the gene sequences of the 7 Campylobacter strains. An appropriate restriction site is then either chosen or introduced by PCR into the gene and a kanamycin resistance cassette is inserted. If the genes of interest are situated in operons (but not at the 3′ end), the kanamycin resistance cassette is used to engineer a non-polar mutation that does not affect the expression of downstream genes. This cassette has been successfully to create a non-polar mutation in the Campylobacter fetus sapC gene (S. A. Thompson, unpublished).

The mutated alleles are introduced into C. jejuni by natural transformation (Bacon et al., 2000 Infect Immun., 68:4384-4390), and the mutations are verified by PCR and hybridization. As mutation recipients, the strains 81-176, 11168, and 81116 are used.

Proteomics experiments are performed comparing the wild-type and isogenic mutants, and those complemented with a wild-type allele on a shuttle plasmid. If the identification of the outer membrane protein is correct, then the single predicted protein spot should disappear in the mutant. If any of these genes play a regulatory role, then proteomics might allow definition of regulatory networks that can be explored in future experiments for understanding C. jejuni. The loss of the outer membrane protein of interest is also verified by immunoblots using polyclonal or monoclonal antibodies.

### Example 6

Generation of Immunologic Reagents to Outer Membrane Proteins

Recombinant outer membrane proteins (rOMPs) are prepared in order to prepare immunologic reagents used to detect outer membrane proteins of interest. This is done by cloning a representative, conserved outer membrane protein-encoding gene into the expression vector pMAL-2, using the pMAL kit from New England Biolabs. This creates protein fusions of the outer membrane proteins of interest with maltose binding protein (MBP), allowing the fusion proteins to be purified by affinity binding to amylose columns. The MBP system has the tremendous potential advantage of greatly increased solubility of the MBP-outer membrane protein fusion proteins (Kapust & Waugh, 1999, Protein Sci., 8:1668-1674), a feature that may be extremely important when dealing with hydrophobic outer membrane proteins. The MBP portion of the purified fusion protein is enzymatically cleaved with a designed protease site in pMAL to test for the solubility of the native rOMP. If the cleaved rOMP is soluble, it is used as the antigen for immunizing rabbits and subsequent experiments. In the event that it is insoluble following cleavage of MBP, the MBP-outer membrane protein fusion is used as the antigen. MBP fusions often allow native folding of the attached proteins (Kapust & Waugh, 1999, Protein Sci., 8:1668-1674); a fusion of MBP with C. coli FlaA (MBP-FlaA) was not only immunogenic, but also protective against Campylobacter disease in mice (Lee et al., 1999 Infect Immun., 67:5799-5805).

Alternatively, His¹⁰-tagged outer membrane protein fusions are constructed using pET16b (Novagen). Fusion proteins are purified from urea lysates of E. coli BL21 (DE3) expressing the rOMP by nickel affinity chromatography according to manufacturer's directions (Qiagen).

rOMPs used for subsequent protection studies are also tested for the ability to induce anti-ganglioside antibodies. Anti-ganglioside antibodies are thought to be important in the pathogenesis of Campylobacter-induced GBS and these rOMPs should not be capable of inducing such antibodies.

C3H/HeN mice are used for analysis of the ganglioside response following immunization with recombinant protein antigens. The strain of mouse selected is based on the observations by Goodyear et al. that mice with this background had good antibody responses to C. jejuni containing ganglioside-mimicry (Goodyear et al., 1999 J Clin Invest., 104:697-708). The RIBI adjuvant system (Corixa Corporation, Seattle, Wash.) is used according to manufacturer's recommendations. An ELISA is used for measuring antiganglioside antibodies according to Willison et al. (1993 J Neurol Sci., 114:209-215). Briefly, gangliosides, GM1, GD1a, GD1b, GD3, GQ1b are purchased from Sigma Chemical Company. Immunolon 2 microplates are coated with 200 ng ganglioside in methanol. Plates are blocked with 2% BSA in PBS. Peroxidase labeled anti-mouse IgG and IgM are used as secondary reagents. Anti-ganglioside antibodies are regularly produced in mice immunized with either whole cell antigens or LOS with the RIBI system and should be a good measure of the ability of recombinant antigens to elicit such antibodies.

Expressed rOMPs are also tested for the ability to exhibit ganglioside mimicry. HRP-labelled cholera toxin and antibodies to different gangliosides (such as GD1a) are used as previously described (Nachamkin et al., 2002 Infect Immun., 70:5299-5303).

Polyclonal rabbit antiserum against the purified recombinant MBP-outer membrane protein fusion proteins are commercially prepared. Polyclonal antiserum recognize many epitopes and will detect conservation of the whole outer membrane protein, not just a specific epitope. Furthermore, polyclonal sera may contain antibodies that recognize conformational epitopes rather than simple linear ones, provided the immunogen is at least partially folded in native conformation.

Recombinant outer membrane proteins or MBP-outer membrane protein fusion proteins (if required for solubility) are used to immunize mice for the production of hybridomas and monoclonal antibodies (Mabs). Mabs are produced using standard protocols well known to one of ordinary skill in the art. Hybridoma supernatants are screened to determine those secreting antibodies against the purified outer membrane protein or MBP-outer membrane protein fusion protein. Those that react with MBP-outer membrane protein are tested in Western blot format using cleaved MBP-OMP as the antigen, to discriminate between Mabs that bind the outer membrane protein and those that bind the MBP portion.

To verify that the outer membrane proteins of interest are actually present in the outer membranes of Campylobacter cells immunogold electron microscopy (EM) is used. EM studies examine surface exposure of the outer membrane proteins in intact whole cells or localization of the outer membrane protein in thin sections of Campylobacter cells. Briefly, intact cells or thin sections of C. jejuni and C. coli strains are washed and incubated with inactivated goat serum to prevent non-specific binding, then treated with either polyclonal antiserum or Mabs recognizing Campylobacter outer membrane proteins. Primary antibody binding is detected using goat anti-rabbit or anti-mouse, respectively, IgG-gold complex (10 nm particles), followed by transmission EM. Gold particle binding to whole cell preparations indicates that the outer membrane protein is outer membrane localized and surface exposed; gold particle binding to the outer membranes of thin sections alone and not to intact cells indicates that the outer membrane protein is located in the outer membrane but not surface exposed. If the gold particles are found in a location not in the outer membrane, it indicates that the original identification of the protein as an outer membrane protein was in error, and that its presence in outer membrane preparations was due to non-specific contamination.

### Example 7

Conservation of OMP-Encoding Genes in C. jejuni Clinical Isolates.

The degree to which the outer membrane proteins are conserved in a much larger bank of C. jejuni strains composed of new clinical isolates and the archival C. jejuni strains is determined.

New clinical isolates are isolated from patients with confirmed Campylobacter infection. Patients agreeing to participate in the study donate 10 to 15 cc of blood by standard venipuncture during the first week of illness (acute) and again at 3 to 4 weeks (convalescent) following the initial onset of symptoms. Blood samples are processed on the day of collection. Sera is stored in aliquots at −70° C. until used for the study.

Campylobacter isolates in the laboratory are identified using a simple biochemical scheme according to previously published methods (Nachamkin, 1999 p. 716-726, In Murray et al., (ed.), Manual of Clinical Microbiology. ASM Press, Washington, D.C.). Routine methods in place at each laboratory are used to isolate and identify Campylobacter spp. All isolates are identified to species level using conventional phenotypic methods and molecular tests; additional tests included hippuricase (Nachamkin, 1999 p. 716-726, In Murray et al., (ed.), Manual of Clinical Microbiology. ASM Press, Washington, D.C.), hipO PCR (Hani & Chan, 1995 J Bacteriol., 177:2396-2402), and species-specific PCR analysis (Gonzalez et al., 1997 J Clin Microbiol., 35:759-763; McIlhinney et al., 1998 J Neurosci Meth., 22:189-194; Oyarzabal et al., 1997 Vet Microbiol., 58:61-71). Isolates are stored at −70° C. in BHI medium with 15% glycerol. Only isolates identified as C. jejuni subsp. jejuni are further characterized.

Isolates are further characterized by serotyping using the Penner serotyping method (Penner & Hennessy, 1980 J Clin Microbiol., 12:732-737). The Nachamkin Laboratory has produced Penner antisera to the 25 most common serotypes observed in the U.S. (Nachamkin et al., 1996 J Clin Microbiol., 34:277-281) as well as additional types associated with Guillain-Barré syndrome. flaA-RFLP analysis is performed according to methods previously described (Nachamkin et al., 1996 J Clin Microbiol., 34:277-281), using a modified consensus primer set described by Wassenaar et al. (Wassenaar & Newell, 2000 Appl Environ Microbiol., 66:1-9).

A substantial strain collection consisting of >400 Campylobacter isolates from patients with gastroenteritis as well as from well-characterized GBS patients (>30 isolates) has been generated. It is first determined whether the genes encoding these outer membrane proteins are present in a large number of C. jejuni strains, using DNA hybridization on dot blots of a large bank of C. jejuni isolates. Chromosomal DNA is purified from each of the C. jejuni strains, and is then arrayed onto nitrocellulose filters in 96-well dot blot format. The 7 Campylobacter strains from Example 3 are included on each filter as positive hybridization controls, and E. coli and H. pylori DNA are included as negative controls. DNA probes are prepared by PCR amplification of the outer membrane protein-encoding genes of interest from one or more of the 7 Campylobacter strains from Example 3. The probes are labeled and hybridized against the filters of C. jejuni DNA, and strains that hybridize with the probe are deemed to possess the outer membrane protein-encoding gene. If there are reproducible differences in the intensity of the hybridization signals with different strains, this may indicate that there is some degree of divergence of the more poorly hybridizing DNA. The proportion of C. jejuni strains that have each outer membrane protein-encoding gene is compiled as an indication of how broadly prevalent the outer membrane protein-encoding gene is among many heterologous strains.

If the outer membrane protein-encoding genes are present in a large number of strains, the degree to which their corresponding primary amino acid sequences are conserved is determined. This is achieved by using PCR amplification and DNA sequencing to characterize the sequences of the genes (and their predicted proteins) encoding 10 or fewer of the most promising candidate outer membrane proteins in a set of Campylobacter strains representing distinct HS serotypes, geographical locations, and MLEE electropherotypes.

Mabs developed against conserved outer membrane proteins are tested for reactivity against clinical Campylobacter isolates. Purified Mabs are labeled with horseradish peroxidase (HRP) according to McIlheny et al. (1998 J Neurosci Meth., 22:189-194). HRP-labeled antibodies are validated for specific reactivity by Western blot analysis with positive and negative control strains and are then used in a dot-blot assay to screen isolates representing a spectrum of serotypes and fla types by methods described previously (Nachamkin et al., 2002 Infect Immun., 70:5299-5303). Mabs that show broad cross-reactivity to Campylobacter isolates are selected for further studies.

The humoral immune response to putative broadly reactive outer membrane proteins is characterized by measuring isotype and subclass specific antibody responses in patients with documented Campylobacter infections. Antibodies against C. jejuni rOMP antigens are measured by an ELISA assay as previously described (Ho et al., 1999 Ann Neurol., 45:168-173). For each recombinant antigen, the assay is optimized with regard to antigen concentration, dilution of secondary antibodies and human sera being assayed. The response to recombinant antigen is compared to the response of a pooled, multicomponent antigen preparation used in previous studies to assess the antibody response in patients with Campylobacter infection (Mishu et al., 1993 Ann Intern Med., 118:947-953).

### Example 8

Protection Against Campylobacter Infection in Mouse Model

Immunization of mice with conserved, purified recombinant outer membrane proteins is performed to determine whether it protects against subsequent experimental C. jejuni infection. Two mouse models have advanced for immunogenic composition studies, involving intranasal immunization followed by either intranasal (“disease”) or oral challenge (colonization).

The two models measure different outcomes. The intranasal model has been used to measure disease (Baqar et al., 1996 Infect Immun., 64:4933-4939; Lee et al., 1999 Infect Immun., 67:5799-5805). Rather than causing diarrhea, the primary disease state in humans, intranasal inoculation of C. jejuni into mice results in pulmonary disease, weight loss, dehydration, ruffled fur, and lethality (Baqar et al., 1996 Infect Immun., 64:4933-4939). Nevertheless, the intranasal challenge model has been used to show the initial protective efficacy of a flagellin immunogenic composition against C. jejuni colonization, as well as disease (Lee et al., 1999 Infect Immun., 67:5799-5805). In contrast, the oral challenge model results in intestinal colonization, much like naturally-occurring colonization of humans. No diarrhea results in these mice, so this model does not measure disease outcomes. The rationale for using both models is that it may be possible to distinguish between different types of protection. Protection in the intranasal infection model amounts to protection against not only colonization, but also systemic spread and disease; the oral challenge model assesses protection only against intestinal colonization.

Lightly anesthetized mice are immunized intranasally with 10-50 μg of rOMP (if rOMP is soluble) or MBP-outer membrane protein fusion protein applied to the external nares as described (Lee et al., 1999 Infect Immun., 67:5799-5805). Immunized mice receive a total of three doses of immunogenic composition or adjuvant only for the control mice 7 days apart. For analysis of the murine immune response to vaccination, intestinal lavage of immunized and control mice is collected at 7 days and whole blood (0.5 ml) is collected 21 days after the last immunization (Baqar et al., 1996 Infect Immun., 64:4933-4939; Lee et al., 1999 Infect Immun., 67:5799-5805). Serum IgA, IgM and IgG, and intestinal (secretory) IgA directed against rOMP are assayed by ELISA. In addition, antiganglioside antibodies are measured as described above.

Immunized and control 6-8 week old BALB/c mice (5-7 mice per group) are challenged orally with homologous (i.e. the strain from which the ROMP was prepared) and heterologous C. jejuni strains as follows. At day 28 following immunization with ROMP, each mouse is pre-treated with 100 μl 10% bicarbonate 30 minutes before feeding a C. jejuni strain grown in BHI-YE medium at 1×10⁷ cfu/mouse (or BHI-YE alone control) with an animal feeding inoculating needle. At day 35, mice are euthanised, the cecum removed, and the cecal contents emptied into pre-weighed tubes. The material is diluted with Muller-Hinton broth to a final concentration at 0.1 g/ml. Ten-fold dilutions are spread onto selective media (CVA Blood agar containing cefoperazone, vancomycin, amphotericin; Becton Dickinson), and incubated at 42° C. in microaerobic conditions for 36 hours or until colonies are discernible. Cecal counts are expressed as CFU/gram of cecal contents.

Cecal colony counts in immunized animals are compared to control animals (immunized with adjuvant only). Data for cecal infection is transformed by taking the log₁₀ of bacteria numbers per gram of cecal contents. Differences in cecal colonization between groups infected with C. jejuni is determined by analysis of variance. Group means are compared with controls using One-way ANOVA (GraphPad Software, Prism, Calif.), with the level of significance set at P<0.05. Immunogenic composition efficacy (% protection) is expressed as ([rate of infection for control mice—rate for vaccinated mice]/rate for control mice)×100.

Intranasal challenge of BALB/c mice is performed as described previously (Baqar et al., 1996 Infect Immun., 64:4933-4939; Lee et al., 1999 Infect Immun., 67:5799-5805). Briefly, C. jejuni strains grown in BHI-YE at 2×10⁹ cfu/mouse (or BHI-YE alone control) are applied to the external nares of lightly anesthetized immunized or control mice (5-7 mice per group). Disease symptoms (health, illness or death) are monitored daily for 5 days post-inoculation, and a daily illness index is calculated. Fecal excretion of C. jejuni is monitored for 14 days post-challenge by culturing 5% fecal homogenates onto CVA agar. Group means are compared with controls using One-way ANOVA.

1. An immunogenic composition comprising a polypeptide encoded by a polynucleotide sequence as defined in any one of
a) SEQ ID NO:1, SEQ ID NO:2, SEQ ID NO:3, SEQ ID NO:4, SEQ ID NO:5, SEQ ID NO:6, SEQ ID NO:8, or SEQ ID NO:10;
b) a polynucleotide sequence encoding the amino acid sequence of SEQ ID NO:7;
c) a polynucleotide sequence encoding the amino acid sequence of SEQ ID NO:9;
d) a polynucleotide sequence encoding the amino acid sequence of SEQ ID NO:11;
e) a polynucleotide sequence encoding the amino acid sequence of SEQ ID NO:12;
f) a polynucleotide sequence encoding the amino acid sequence of SEQ ID NO:13;
g) a polynucleotide sequence encoding the amino acid sequence of SEQ ID NO:14;
h) a polynucleotide of at least 50 consecutive nucleotides of any of a)-g); and
i) an ortholog or homolog of any of a)-g).

2. The immunogenic composition of claim 1, wherein the polynucleotide sequence is defined in any of SEQ ID NO:1, SEQ ID NO:2, SEQ ID NO:3, SEQ ID NO:4, SEQ ID NO:5, or SEQ ID NO:6.

3. The immunogenic composition of claim 1, wherein the polynucleotide sequence is defined in SEQ ID NO:8.

4. The immunogenic composition of claim 1, wherein the polynucleotide sequence is defined in SEQ ID NO:10.

5. The immunogenic composition of claim 1, wherein the polypeptide is defined in SEQ ID NO:7.

6. The immunogenic composition of claim 1, wherein the polypeptide is defined in SEQ ID NO:9.

7. The immunogenic composition of claim 1, wherein the polypeptide is defined in SEQ ID NO:11.

8. The immunogenic composition of claim 1, wherein the polypeptide is defined in SEQ ID NO:12.

9. The immunogenic composition of claim 1, wherein the polypeptide is defined in SEQ ID NO:13.

10. The immunogenic composition of claim 1, wherein the polypeptide is defined in SEQ ID NO:14.

11. An immunogenic composition comprising a purified polypeptide as defined in any one of:
a) SEQ ID NO:7, SEQ ID NO:9, SEQ ID NO:11, SEQ ID NO:12, SEQ ID NO:13, or SEQ ID NO:14;
b) a 50 amino acid fragment of any of a); and
c) an ortholog or homolog of any of a).

12. The immunogenic composition of claim 11, wherein the polypeptide is defined in SEQ ID NO:7.

13. The immunogenic composition of claim 11, wherein the polypeptide is defined in SEQ ID NO:9.

14. The immunogenic composition of claim 11, wherein the polypeptide is defined in SEQ ID NO:11.

15. The immunogenic composition of claim 11, wherein the polypeptide is defined in SEQ ID NO:12.

16. The immunogenic composition of claim 11, wherein the polypeptide is defined in SEQ ID NO:13.

17. The immunogenic composition of claim 11, wherein the polypeptide is defined in SEQ ID NO:14.

18. A live bacterial cell vector that (a) infects a human and (b) is stably transformed with, and expresses, a heterologous DNA encoding antigen EF-Tu, operatively associated with a regulatory sequence that controls gene expression.

19. The vector of claim 18, wherein the heterologous DNA encodes an amino acid as defined in SEQ ID NO:7, a consecutive 50 amino acid fragment thereof, or an ortholog or homolog thereof.

20. A live bacterial cell vector that (a) infects a human and (b) is stably transformed with, and expresses, a heterologous DNA encoding antigen Cj0069, operatively associated with a regulatory sequence that controls gene expression.

21. The vector of claim 20, wherein the heterologous DNA encodes an amino acid as defined in SEQ ID NO:9, a consecutive 50 amino acid fragment thereof, or an ortholog or homolog thereof.

22. A live bacterial cell vector that (a) infects a human and (b) is stably transformed with, and expresses, a heterologous DNA encoding antigen Cj0561c, operatively associated with a regulatory sequence that controls gene expression.

23. The vector of claim 22, wherein the heterologous DNA encodes an amino acid as defined in SEQ ID NO:11, a consecutive 50 amino acid fragment thereof, or an ortholog or homolog thereof.

24. A live bacterial cell vector that (a) infects a human and (b) is stably transformed with, and expresses, a heterologous DNA encoding antigen AstA, operatively associated with a regulatory sequence that controls gene expression.

25. The vector of claim 24, wherein the heterologous DNA encodes an amino acid as defined in SEQ ID NO:12, a consecutive 50 amino acid fragment thereof, or an ortholog or homolog thereof.

26. A live bacterial cell vector that (a) infects a human and (b) is stably transformed with, and expresses, a heterologous DNA encoding antigen Rv2794c, operatively associated with a regulatory sequence that controls gene expression.

27. The vector of claim 26, wherein the heterologous DNA encodes an amino acid as defined in SEQ ID NO:13, a consecutive 50 amino acid fragment thereof, or an ortholog or homolog thereof.

28. A live bacterial cell vector that (a) infects a human and (b) is stably transformed with, and expresses, a heterologous DNA encoding antigen DRC0015, operatively associated with a regulatory sequence that controls gene expression.

29. The vector of claim 28, wherein the heterologous DNA encodes an amino acid as defined in SEQ ID NO:14, a consecutive 50 amino acid fragment thereof, or an ortholog or homolog thereof.

30. A method of eliciting an immune response in an animal, comprising introducing into the animal a composition comprising a purified polypeptide encoded by a polynucleotide sequence as defined in any one of:
a) SEQ ID NO:1, SEQ ID NO:2, SEQ ID NO:3, SEQ ID NO:4, SEQ ID NO:5, SEQ ID NO:6, SEQ ID NO:8, or SEQ ID NO:10;
b) a polynucleotide sequence encoding the amino acid sequence of SEQ ID NO:7;
c) a polynucleotide sequence encoding the amino acid sequence of SEQ ID NO:9;
d) a polynucleotide sequence encoding the amino acid sequence of SEQ ID NO:11;
e) a polynucleotide sequence encoding the amino acid sequence of SEQ ID NO:12;
f) a polynucleotide sequence encoding the amino acid sequence of SEQ ID NO:13;
g) a polynucleotide sequence encoding the amino acid sequence of SEQ ID NO:14;
h) a polynucleotide of at least 50 consecutive nucleotides of any of a)-g); and
i) an ortholog or homolog of any of a)-g).

31. The method of claim 30, wherein the purified polypeptide is encoded by a polynucleotide sequence as defined in any one of SEQ ID NO:1, SEQ ID NO:2, SEQ ID NO:3, SEQ ID NO:4, SEQ ID NO:5, or SEQ ID NO:6.

32. The method of claim 30, wherein the purified polypeptide is encoded by a polynucleotide sequence as defined in SEQ ID NO:8.

33. The method of claim 30, wherein the purified polypeptide is encoded by a polynucleotide sequence as defined in SEQ ID NO:10.

34. The method of claim 30, wherein the purified polypeptide is defined in SEQ ID NO:7.

35. The method of claim 30, wherein the purified polypeptide is defined in SEQ ID NO:9.

36. The method of claim 30, wherein the purified polypeptide is defined in SEQ ID NO:11.

37. The method of claim 30, wherein the purified polypeptide is defined in SEQ ID NO:12.

38. The method of claim 30, wherein the purified polypeptide is defined in SEQ ID NO:13.

39. The method of claim 30, wherein the purified polypeptide is defined in SEQ ID NO:14.

40. A method of generating antibodies specific for antigen EF-Tu, comprising introducing into an animal a composition comprising a purified polypeptide encoded by a polynucleotide sequence as defined in any one of:
a) SEQ ID NO:1, SEQ ID NO:2, SEQ ID NO:3, SEQ ID NO:4, SEQ ID NO:5, or SEQ ID NO:6;
b) a polynucleotide sequence encoding the amino acid sequence of SEQ ID NO:7;
c) a polynucleotide of at least 50 consecutive nucleotides of any of a) or b); and
d) an ortholog or homolog of any of a) or b).

41. The method of claim 40, wherein the purified polypeptide is encoded by a polynucleotide sequence as defined in any one of SEQ ID NO:1, SEQ ID NO:2, SEQ ID NO:3, SEQ ID NO:4, SEQ ID NO:5, or SEQ ID NO:6.

42. The method of claim 40, wherein the purified polypeptide is defined in SEQ ID NO:7.

43. A method of generating antibodies specific for antigen Cj0069, comprising introducing into an animal a composition comprising a purified polypeptide encoded by a polynucleotide sequence as defined in any one of:
a) SEQ ID NO:8;
b) a polynucleotide sequence encoding the amino acid sequence of SEQ ID NO:9;
c) a polynucleotide of at least 50 consecutive nucleotides of any of a) or b); and
d) an ortholog or homolog of any of a) or b).

44. The method of claim 43, wherein the purified polypeptide is encoded by a polynucleotide sequence as defined in SEQ ID NO:8.

45. The method of claim 43, wherein the purified polypeptide is defined in SEQ ID NO:9.

46. A method of generating antibodies specific for antigen Cj0561c, comprising introducing into an animal a composition comprising a purified polypeptide encoded by a polynucleotide sequence as defined in any one of:
a) SEQ ID NO:10;
b) a polynucleotide sequence encoding the amino acid sequence of SEQ ID NO:11;
c) a polynucleotide of at least 50 consecutive nucleotides of any of a) or b); and
d) an ortholog or homolog of any of a) or b).

47. The method of claim 46, wherein the purified polypeptide is encoded by a polynucleotide sequence as defined in SEQ ID NO:10.

48. The method of claim 46, wherein the purified polypeptide is defined in SEQ ID NO:11.

49. A method of generating antibodies specific for antigen AstA, comprising introducing into an animal a composition comprising a purified polypeptide as defined in the amino acid sequence of SEQ ID NO:12, a consecutive 50 amino acid fragment thereof, or an ortholog or homolog thereof.

50. A method of generating antibodies specific for antigen Rv2794c, comprising introducing into an animal a composition comprising a purified polypeptide as defined in the amino acid sequence of SEQ ID NO:13, a consecutive 50 amino acid fragment thereof, or an ortholog or homolog thereof.

51. A method of generating antibodies specific for antigen DRC0015, comprising introducing into an animal a composition comprising a purified polypeptide as defined in the amino acid sequence of SEQ ID NO:14, a consecutive 50 amino acid fragment thereof, or an ortholog or homolog thereof.

52. The method of any one of claims 40, 43, 46, 49, 50, or 51, further comprising re-introducing the purified polypeptide to the animal after approximately 1 week.

53. The method of any one of claims 40, 43, 46, 49, 50, or 51, further comprising detecting the presence in the animal of antibodies specific for the antigen.

54. The method of any one of claims 40, 43, 46, 49, 50, or 51, wherein the animal is susceptible to infection with Campylobacter.

55. The method of any one of claims 40, 43, 46, 49, 50, or 51, wherein the amount of the purified polypeptide is sufficient to induce an immune response protective against Campylobacter infection.

56. A purified antibody that binds specifically to a protein selected from the group consisting of EF-Tu, Cj0069, Cj0561c, AstA, Rv2794c, and DRC0015.

57. The antibody of claim 56, wherein the antibody is selected from the group consisting of recombinant antibodies, humanized chimeric antibodies and immunologically active fragments of antibodies.

58. A method of making an antibody, comprising immunizing a non-human animal with an immunogenic fragment of a protein selected from a group consisting of EF-Tu, Cj0069, Cj0561c, AstA, Rv2794c, and DRC0015.

59. A method of making an antibody, comprising providing a hybridoma cell that produces a monoclonal antibody specific for a protein selected from a group consisting of EF-Tu, Cj0069, Cj0561c, AstA, Rv2794c, and DRC0015, and culturing the cell under conditions that permit production of the monoclonal antibody.

60. A method of inhibiting Campylobacter infection in a patient, comprising administering to the patient a composition a purified antibody that binds specifically to a protein selected from a group consisting of EF-Tu, Cj0069, Cj0561c, AstA, Rv2794c, and DRC0015, wherein the antibody is selected from the group consisting of recombinant antibodies, humanized chimeric antibodies and immunologically active fragments of antibodies.

61. A method of determining whether a biological sample contains C. jejuni, comprising contacting the sample with the antibody of claim 56 and determining whether the antibody specifically binds to the sample, said binding being an indication that the sample contains C. jejuni.

62. A method of purifying a protein from a biological sample containing a protein selected from a group consisting of EF-Tu, Cj006, Cj0561c, AstA, Rv2794c, and DRC0015, comprising
a) providing an affinity matrix comprising the antibody of claim 56 bound to a solid support;
b) contacting the biological sample with the affinity matrix, to produce an affinity matrix-protein complex;
c) separating the affinity matrix-protein complex from the remainder of the biological sample; and
d) releasing the protein from the affinity matrix.

63. The method of claim 62, wherein the protein purified from the affinity matrix comprises the amino acid sequence as defined in SEQ ID NO:7, or an ortholog or homolog thereof.

64. The method of claim 62, wherein the protein purified from the affinity matrix comprises the amino acid sequence as defined in SEQ ID NO:9, or an ortholog or homolog thereof.

65. The method of claim 62, wherein the protein purified from the affinity matrix comprises the amino acid sequence as defined in SEQ ID NO:11, or an ortholog or homolog thereof.

66. The method of claim 62, wherein the protein purified from the affinity matrix comprises the amino acid sequence as defined in SEQ ID NO:12, or an ortholog or homolog thereof.

67. The method of claim 62, wherein the protein purified from the affinity matrix comprises the amino acid sequence as defined in SEQ ID NO:13, or an ortholog or homolog thereof.

68. The method of claim 62, wherein the protein purified from the affinity matrix comprises the amino acid sequence as defined in SEQ ID NO:14, or an ortholog or homolog thereof.
